(19) United States
(12) Patent Application Publication
(10) Pub. No.: US 2023/0054593 A1
(43) Pub. Date: Feb. 23, 2023

(54) METHODS OF TREATING FRAGILE X SYNDROME WITH REELIN

(71) Applicants: Edwin WEEBER, Apollo Beach, FL (US); UNIVERSITY OF SOUTH FLORIDA, Tampa, FL (US)

(72) Inventors: Edwin WEEBER, Apollo Beach, FL (US); Kevin R. NASH, Temple Terrace, FL (US); Qingyou LI, Tampa, FL (US); Nicole MORRILL, Tampa, FL (US)

(21) Appl. No.: 17/793,506

(22) PCT Filed: Jan. 19, 2021
(86) PCT No.: PCT/US2021/013960

§ 371 Date: Jul. 18, 2022

Related U.S. Application Data
(60) Provisional application No. 62/962,609, filed on Jan. 17, 2020.

(51) Int. Cl. A61K 38/48; A61P 25/28

(52) U.S. Cl. CPC A61K 38/482; A61P 25/28; A61K 48/00

(57) ABSTRACT

Fragile X syndrome (FXS) is the most common inherited form of human intellectual disability. FXS is caused by loss of function of the FMR1 gene which results in significant behavioral deficits in spatial learning and memory tests. FMR1−/− knockout mice share many of the learning deficits and decreased synaptic function encountered in FXS patients. Anecdotal evidence indicates a reduction in the amount of Reelin, a large extracellular signaling protein important for normal hippocampal synaptic plasticity, may play role in the etiology of FXS. Disclosed herein is a rAAV9 Reelin viral vector expressing a REELIN repeat R3+R6 fusion protein that is shown to rescue cognitive deficits in FMR1−/− mice as evaluated in the Hidden Platform Water Maze, Open Field and Fear Conditioning. Reelin gene therapy is therefore potentially a novel therapeutic for the treatment of Fragile X Syndrome.

This application claims the benefit of U.S. Provisional Application No. 62/962,609, filed on Jan. 17, 2020, the content of which is incorporated by reference herein in its entirety.

### BACKGROUND

Fragile X Syndrome (FXS) is the most common inherited form of intellectual disability and is characterized by childhood seizures, and several Autism Spectrum Disorder (ASD)-like symptoms, including social dysfunction, hyperactivity, stereotypic movements, hand-flapping and hand-biting, speech delay, and a relative lack of expressive language ability. FXS is caused by loss of function mutations in the Fragile X Mental Retardation 1 (FMR1) gene. The subsequent silencing of Fragile X Mental Retardation Protein (FMRP) expression causes disruption to glutamatergic and GABAergic neurotransmission, overall dysregulation of synaptic plasticity and the formation of abnormal immature dendritic spines.

FXS remains an incurable disease. Current research aims at treating the cognitive deficits caused by this devastating disease. FMR1 knockout mice provide a useful animal model for testing the efficacy of potential FXS therapeutics primarily because it reproduces several aspects of the FXS phenotype including learning deficits and decreased synaptic function. The extracellular matrix protein REELIN (RELN) has been implicated in numerous neurological disorders, including Fragile X Syndrome, schizophrenia, bipolar disorder, depression, autism, and Alzheimer's disease (AD).

Reelin is proteolytically processed in vivo between EGF-like repeats 2-3 (R2-3) and repeats 6-7 (R6-7). These cleavage sites produce five major fragments that can be found in the adult and developing brain. The middle REELIN repeat R3-6 fragment interacts with the VLDLR and ApoER2 and initiates downstream signaling of the Reelin cascade. However, a viable REELIN therapeutic for the treatment of Fragile X Syndrome has yet to make it to market.

### SUMMARY

Disclosed are methods and compositions related to the treatment of Fragile X Syndrome through the administration of a recombinant REELIN protein fragment or REELIN protein fragment encoded by a spliced REELIN mRNA transcript (referred to herein as a native REELIN protein fragment) that act upon the lipoprotein receptor system in a manner similar to native REELIN.

In one aspect disclosed herein are methods of treating, inhibiting, reducing, decreasing, and/or ameliorating Fragile X Syndrome, comprising administering a therapeutically effective amount of a recombinant REELIN protein fragment or native REELIN protein fragment into a patient in need thereof; wherein the recombinant REELIN protein fragment is selected from the group consisting of a REELIN repeat R3 protein fragment, a REELIN repeat R4 protein fragment, a REELIN repeat R5 protein fragment, a REELIN repeat R6 protein fragment, a REELIN repeat R3 through R4 protein fragment, a REELIN repeat R3 through R5 protein fragment, a REELIN repeat R3 through R6 protein fragment, a REELIN repeat R4 through R5 protein fragment, a REELIN repeat R5 through R6 protein fragment, a REELIN repeat R3 joined to repeat R5 protein fragment, REELIN repeat R3 joined to a repeat R6 protein fragment, REELIN repeat R4 joined to a repeat R6 protein fragment, or combinations thereof; wherein R3 is repeat region 3 of full length REELIN, R4 is repeat region 4 of full length REELIN, R5 is repeat region 5 of full length REELIN, and R6 is repeat region 6 of full length REELIN.

Also disclosed herein are methods of treating, inhibiting, reducing, decreasing, and/or ameliorating a symptom of Fragile X Syndrome, (such as, for example, deficiency in dendritic spine density, diminished long-term potentiation, diminished synaptic plasticity and associative learning deficits), comprising: administering a therapeutically effective amount of a recombinant REELIN protein fragment or native REELIN protein fragment into a patient in need thereof; wherein the recombinant REELIN protein fragment is selected from the group consisting of a REELIN repeat R3 protein fragment, a REELIN repeat R4 protein fragment, a REELIN repeat R5 protein fragment, a REELIN repeat R6 protein fragment, a REELIN repeat R3 through R4 protein fragment, REELIN repeat R3 through R5 protein fragment, REELIN repeat R3 through R6 protein fragment, REELIN repeat R4 through R5 protein fragment, REELIN repeat R5 through R6 protein fragment, REELIN repeat R3 joined to a repeat R5 protein fragment, REELIN repeat R3 joined to a repeat R6 protein fragment, REELIN repeat R4 joined to a repeat R6 protein fragment, or combinations thereof; wherein R3 is repeat region 3 of full length REELIN, R4 is repeat region 4 of full length REELIN, R5 is repeat region 5 of full length REELIN, and R6 is repeat region 6 of full length REELIN. Thus, in one aspect, disclosed herein are methods of treating, inhibiting, reducing, decreasing, and/or ameliorating at least one symptom (such as, for example, dendritic spine density, diminished long-term potentiation, diminished synaptic plasticity, hyperactivity, spatial learning, memory, and/or associative learning deficits) of a patient suffering from Fragile X Syndrome, comprising administering (for example, by intraarterially, intravenously, intracerebrally (including, but not limited to bilateral intracerebral injection or intracerebroventricular (ICV) injection), intraventricularly or intrathecally) a therapeutically effective amount of a REELIN adeno-associated viral (AAV) vector expressing a secreted recombinant REELIN fusion protein, wherein the REELIN fusion protein comprises an N-terminal REELIN R3 repeat encoded by the nucleotide sequence of SEQ ID NO: 2. In some aspects, the recombinant REELIN fusion protein does not comprise a REELIN repeat R4 and R5.

Also disclosed herein are methods of treating, inhibiting, reducing, decreasing, and/or ameliorating at least one symptom of a patient suffering from Fragile X Syndrome of any preceding aspect, wherein the recombinant REELIN fusion protein further comprises a C-terminal REELIN R6 repeat encoded by the nucleotide sequence of SEQ ID NO: 5 and/or an IgKappa signal sequence fused in frame to the N-terminal REELIN R3 repeat. For example, disclosed herein are of treating, inhibiting, reducing, decreasing, and/or ameliorating at least one symptom of a patient suffering from Fragile X Syndrome, wherein the recombinant REELIN fusion protein has the amino acid sequence of SEQ ID NO: 10.

In one aspect, disclosed herein are methods of treating, inhibiting, reducing, decreasing, and/or ameliorating at least one symptom of a patient suffering from Fragile X Syndrome of any preceding aspect, wherein the REELIN AAV vector is effective at mitigating at least one cognitive defect caused by Fragile X Syndrome (such as, for example, hyperactivity, associative learning, spatial learning and memory).

Also disclosed herein are methods of treating, inhibiting, reducing, decreasing, and/or ameliorating at least one symptom of a patient suffering from Fragile X Syndrome of any preceding aspect, wherein the mitigation of the at least one cognitive defect caused by Fragile X Syndrome is equivalent to the mitigation of the cognitive defect obtained after intracerebroventricular (ICV) injection of a R3456 REELIN repeat protein.

In one aspect, disclosed herein are methods of treating, inhibiting, reducing, decreasing, and/or ameliorating at least one symptom of a patient suffering from Fragile X Syndrome of any preceding aspect, wherein the recombinant REELIN fusion protein induces dimerization of an ApoER2 receptor and/or activates the phosphorylation of DAB1 and ERK1/2.

Disclosed herein are methods of treating, inhibiting, reducing, decreasing, and/or ameliorating at least one symptom (such as, for example, dendritic spine density, diminished long-term potentiation, diminished synaptic plasticity, hyperactivity, spatial learning, memory, and/or associative learning deficits) of a patient suffering from Fragile X Syndrome, comprising administering a therapeutically effective amount of a REELIN adeno-associated viral (AAV) vector expressing a secreted recombinant REELIN fusion protein, wherein the fusion protein comprises a C-terminal R6 REELIN repeat encoded by the nucleotide sequence of SEQ ID NO: 5. In some aspects, the recombinant REELIN fusion protein does not comprise a REELIN repeat R4 and R5

A method of treating at least one symptom of a patient suffering from Fragile X Syndrome, comprising administering a therapeutically effective amount of a REELIN adeno-associated viral (AAV) vector expressing a secreted recombinant REELIN fusion protein, wherein the REELIN fusion protein does not comprise REELIN repeats R4 or R5.

In one aspect, disclosed herein are methods of treating, inhibiting, reducing, decreasing, and/or ameliorating Fragile X Syndrome of any preceding aspect and methods of treating, inhibiting, reducing, decreasing, and/or ameliorating a symptom of Fragile X Syndrome of any preceding aspect, wherein the REELIN protein fragment is administered at between 1 μl and 2 μl of a 5 nM composition for each 30-36 g of patient mass.

Also disclosed herein in one aspect, are methods of treating, inhibiting, reducing, decreasing, and/or ameliorating Fragile X Syndrome of any preceding aspect and methods of treating, inhibiting, reducing, decreasing, and/or ameliorating a symptom of Fragile X Syndrome of any preceding aspect, wherein the recombinant REELIN protein fragment or native REELIN protein fragment can be injected intraarterially, intravenously, intracerebrally, intraventricularly or intrathecally. For example, the recombinant REELIN protein fragment can be bilaterally injected into the brain (e.g., a bilateral intracerebral injection) of a patient in need thereof. In certain aspects, the recombinant REELIN protein fragment or native REELIN protein fragment can be injected into one or more specific regions of the brain, including, for example, the cortex, hippocampus, thalamus, hypothalamus, cerebellum, brain stem or spinal cord.

In one aspect, disclosed herein are methods of treating, inhibiting, reducing, decreasing, and/or ameliorating Fragile X Syndrome of any preceding aspect and methods of treating, inhibiting, reducing, decreasing, and/or ameliorating a symptom of Fragile X Syndrome of any preceding aspect, further comprising inserting an expression construct encoding a recombinant REELIN protein fragment or native REELIN protein fragment into a viral vector (such as, for example, a viral vector selected from the group consisting of AAV-9, AAV-5, AAV-4, AAV-2 and AAV-1 or any variant thereof) to form a REELIN viral vector; and injecting the REELIN vector intraarterially, intravenously, intracerebrally, intraventricularly or intrathecally into a subject. In certain aspects, the viral vector expresses a recombinant REELIN protein fragment or native REELIN protein fragment; wherein the recombinant REELIN protein fragment is selected from the group consisting of a REELIN repeat R3 protein fragment, a REELIN repeat R4 protein fragment, a REELIN repeat R5 protein fragment, a REELIN repeat R6 protein fragment, a REELIN repeat R3 through R4 protein fragment, a REELIN repeat R3 through R5 protein fragment, a REELIN repeat R3 through R6 protein fragment, a REELIN repeat R4 through R5 protein fragment, a REELIN repeat R5 through R6 protein fragment, a REELIN repeat R3 joined to repeat R5 protein fragment, a REELIN repeat R3 joined to repeat R6 protein fragment, a REELIN repeat R4 joined to repeat R6 protein fragment, or combinations thereof; wherein R3 is repeat region 3 of full length REELIN, R4 is repeat region 4 of full length REELIN, R5 is repeat region 5 of full length REELIN, and R6 is repeat region 6 of full length REELIN. In some aspects, the REELIN viral vector can be injected into one or more specific regions of the brain, including, for example, the cortex, hippocampus, thalamus, hypothalamus, cerebellum, brain stem or spinal cord. In some aspects, the expression of the recombinant REELIN fragment is under the control of a eukaryotic promotor, e.g., a CMV promoter.

Also disclosed herein are compositions for using in any of the methods of treating, inhibiting, reducing, decreasing, and/or ameliorating Fragile X Syndrome of any preceding aspect and methods of treating, inhibiting, reducing, decreasing, and/or ameliorating a symptom of a Fragile X Syndrome of any preceding aspect.

In one aspect, disclosed herein are compositions comprising a recombinant REELIN protein fragment or a native REELIN protein fragment; wherein the recombinant REELIN protein fragment is selected from the group consisting of a REELIN repeat R3 protein fragment, a REELIN repeat R4 protein fragment, a REELIN repeat R5 protein fragment, a REELIN repeat R6 protein fragment, a REELIN repeat R3 through R4 protein fragment, a REELIN repeat R3 through R5 protein fragment, a REELIN repeat R3 through R6 protein fragment, a REELIN repeat R4 through R5 protein fragment, a REELIN repeat R5 through R6 protein fragment, a REELIN repeat R3 joined to repeat R5 protein fragment, a REELIN repeat R3 joined to repeat R6 protein fragment, a REELIN repeat R4 joined to repeat R6 protein fragment, or combinations thereof; wherein R3 is repeat region 3 of full length REELIN, R4 is repeat region 4 of full length REELIN, R5 is repeat region 5 of full length REELIN, and R6 is repeat region 6 of full length REELIN.

In another aspect, disclosed herein are compositions comprising a REELIN viral vector (such as, for example, a viral vector selected from the group consisting of AAV-9, AAV-5, AAV-4, AAV-2 and AAV-1 or any variant thereof) having a transgene for expressing a recombinant REELIN protein fragment or a native REELIN protein fragment, wherein the recombinant REELIN protein fragment is selected from the group consisting of a REELIN repeat R3 protein fragment, a REELIN repeat R4 protein fragment, a REELIN repeat R5 protein fragment, a REELIN repeat R6 protein fragment, a REELIN repeat R3 through R4 protein fragment, a REELIN repeat R3 through R5 protein fragment, a REELIN repeat R3 through R6 protein fragment, a REELIN repeat R4 through R5 protein fragment, a REELIN repeat R5 through R6 protein fragment, a REELIN repeat R3 joined to repeat R5 protein fragment, a REELIN repeat R3 joined to repeat R6 protein fragment, a REELIN repeat R4 joined to repeat R6 protein fragment, or combinations thereof; wherein R3 is repeat region 3 of full length REELIN, R4 is repeat region 4 of full length REELIN, R5 is repeat region 5 of full length REELIN, and R6 is repeat region 6 of full length REELIN.

In some aspects, the REELIN viral vector can be injected into one or more specific regions of the brain, including, for example, the cortex, hippocampus, thalamus, hypothalamus, cerebellum, brain stem or spinal cord. In some aspects, the expression of the recombinant REELIN fragment can be under the control of a eukaryotic promotor, e.g., a CMV promoter.

### BRIEF DESCRIPTION OF THE DRAWINGS

The accompanying drawings, which are incorporated in and constitute a part of this specification, illustrate several embodiments and together with the description illustrate the disclosed compositions and methods.

FIG. 1A shows a diagram of REELIN (RELN) protein including novel construct. Arrows indicate in vivo cleavage sites.

FIG. 1B shows an exemplary Reelin signaling pathway.

FIG. 1C is a Western blot showing a significant decrease in REELIN in FMR1−/− mice compared to wild type controls.

FIG. 1D depicts the location of REELIN repeat protein fragments within full length REELIN protein. SS: IgKappa signal sequence

FIGS. 2A, 2B, 2C, 2D, and 2E show a REELIN binding assay. FIG. 2A shows both the recombinant R3456 and R36 REELIN repeat protein fragments can bind to the LRP8 receptor as determined by luciferase activity (2B) and activate signal transduction in primary neurons: DAB1 phosphorylation (2C) and ERK phosphorylation (2D) when compared to a no RELN control (CTL) and a RELN N-terminal fragment (NR2) control. FIG. 2E shows a Western blot for pDAB1, pERK1/2 and actin. ANOVA, Tukey post-hoc. *p<0.05. n=4 replicates per test.

FIGS. 3A, 3B, and 3C show that injection of the recombinant R3456 REELIN repeat protein fragment (also referred to as R3-6) into FXS mice (FMR1 KO) rescues neurological deficits. FIG. 3A shows the partial rescue of hyperactivity in FMR1 KO mice after injection of the R3-6 REELIN repeat protein fragment. FIG. 3B shows injection of the R3-R6 REELIN repeat protein fragment into FXS mice reduced recall in the contextual fear conditioning test. FIG. 3C shows that injection of R3-R6 REELIN repeat protein fragment into FXS mice also rescues Morris water maze deficits during a probe trail at 72 h post-training. Mice were allowed to recover for two days prior to initiating behavioral testing n=7/group. ANOVA with Tukey post-hoc. ***p<0.001.

FIGS. 4A, 4B, and 4C show that injection of a R3-R6 REELIN repeat protein fragment into FMR1−/− mice rescues the general locomotor activity deficit observed in an open-field maze test (OFM). FMR1−/− mice were observed in the OFM 72 HR after injection of either a mock or the R3-R6 REELIN repeat protein fragment. FIG. 4A shows the R3-R6 REELIN repeat protein fragment significantly reduced total ambulatory distance in FMR1−/− mice as compared to mock injected FMR1−/− mice and injected Tyr wild type controls. FIG. 4B shows FMR1−/− mice injected with the R3-R6 REELIN repeat protein fragment spent significantly less time in the center of the OFM as compared to mock injected FMR1−/− mice. In FIG. 4C, FMR1−/− mice injected with the R3-R6 REELIN repeat protein fragment showed a decrease in fecal boli when compared to mock injected FMR1−/− mice. A significant difference was also observed between the mock injected FMR1−/− mice and Tyr wild type mice. For all groups, n=7. *p<0.05.

FIGS. 5A, 5B, 5C, and 5D show the R3-R6 REELIN repeat protein fragment supplementation enhanced spatial learning and memory in FMR1−/− mice. FIGS. 5A and 5B show R3-R6 REELIN repeat protein fragment injected FMR1−/− mice spent a significantly greater amount of time in the target quadrant during the 24 h (5A) and 72 h (5B) probe trial compared to mock-treated FMR1−/− animals. A significant difference was also observed between FMR1−/− mock injected mice and Tyr wild-type mock injected mice. FIG. 5C shows R3-R6 REELIN repeat protein fragment injected FMR1−/− mice showed significantly increased target platform crosses during the 24 h probe trial compared to mock-treated FMR1−/− animals. A significant difference was also observed between FMR1−/− mock injected mice and Tyr wild-type mock injected mice. FIG. 5D shows no significant improvement in platform crosses was observed at 72 h probe trial for R3-R6 REELIN repeat protein fragment injected FMR1−/− mice. For all groups, n=7. *p<0.05.

FIGS. 6A and 6B show that injection of R3-R6 REELIN repeat protein fragment recovers the associative learning deficit observed in Fmr1−/− mice. (A) Freezing to the Context was assessed for 3 min 24 h post-training. Reelin supplementation increased the average context-dependent freezing in REELIN injected FMR1−/− mice compared to mock injected FMR1−/− and Tyr wild-type mice. (B) There were no significant differences in freezing to the cue between experimental groups at 24 h. For all groups, n=7.

FIGS. 7A, 7B, and 7C show that injection of the R36 REELIN repeat protein fragment (also referred to herein as R3+6 and R3+R6) and rAAV9-R36 in FXS (FMR1 KO) mice rescues neurological deficits. FIG. 7A shows rescued hyperactivity, FIG. 7B shows rescue of reduced recall in contextual fear conditioning and FIG. 7C shows the rescue of Morris water maze deficits during probe trails at 72 h post training. n=7-8/group. ANOVA with Tukey post-hoc. ***p<0.001.

FIGS. 8A and 8B show six weeks of rAAV9-R3+6 expression in wild type (WT) mice. FIG. 8A shows no detrimental effect of rAAV9-R3+6 in the open field test as compared to wild type mice injected with the rAAV9-GFP control. FIG. 8B shows a statistically significant improvement in the Morris water maze (MWM) during probe trails at 72 h post training with rAAV9-R3+6 as compared to wild type mice injected with the rAAV9-GFP control. n=10/group. Student's t test ***p<0.001.

FIG. 9A shows a map of the expression plasmid pSEC R3+R6 and the pTR-R3+R6 viral vector. CBA: chicken beta actin promoter; Ig-kappa signal: Immunoglobulin kappa signal sequence; WPRE: Woodchuck Hepatitis Virus (WHP) Posttranscriptional Regulatory Element;

FIG. 9B shows a Reelin fragment R3-6 expression plasmid.

FIG. 9C shows R3+6 REELIN repeat protein expression in HEK293 cells transfected with either a control plasmid, pTR-R3+R6 viral vector or pSEC R3+R6 expression plasmid. A dot blot of myc-tagged R3+6 REELIN repeat protein fragment in cell lysate and in cell culture medium was detected using an anti-myc antibody.

FIGS. 10A, 10B and 10C show the distribution of AAV vectors after injection. FIG. 10A shows intracerebroventricular (ICV) injection of AAV9 results in vector distribution throughout the rat central nervous system (CNS) projecting into cortical, hippocampal, and striatal regions (n=3) (n=10(4F/6M)); ACX: anterior cortex, PCX: posterior cortex, HPC: hippocampus, STR: striatum, CER: cerebellum, THA: thalamus. FIG. 10B compares GFP staining of AAV-GFP serotypes 2, 7, 8 and 9 injected into a mouse thalamus. Injection of AAV9 into the thalamus resulted in GFP staining throughout the mouse brain. *p<0.05 compared to AAV-2.

### DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

Titles or subtitles may be used in the specification for the sole convenience of the reader but are not intended to influence the scope of the present disclosure or to limit any aspect of the disclosure to any subsection, subtitle, or paragraph.

### Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

As used herein, the term “Reelin repeat” refers to one of 8 repeating units which consist of an amino acid sequence with an EGF-like motif at its center, and which are homologous to one other. REELIN repeats R3, R4, R5 and R6 have the following amino acid sequences:

| | | N-TERMINAL AMINO ACID |
|---|---|---|
| | NUCLEOTIDE | RESIDUE TO C-TERMINAL |
| REELIN | SEQUENCE | AMINO ACID RESIDUE OF |
| REPEAT | SEQ ID NO | FULL LENGTH REELIN |
| R3 | 2 | 1313-1555 |
| R4 | 3 | 1675-1904 |
| R5 | 4 | 2032-2270 |
| R6 | 5 | 2388-2618 |

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. Thus, treatment includes obtaining beneficial or desired clinical results. Beneficial or desired clinical results include, but are not limited to, any one or more of: alleviation of one or more symptoms, diminishment of extent of fragile X syndrome, stabilization (i.e., not worsening) of the state of the neurodegenerative disease or neurological insult, preventing or delaying occurrence or recurrence of fragile X syndrome, delay or slowing of disease progression and amelioration of the disease state. The methods of the invention contemplate any one or more of these aspects of treatment.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or “mitigate” or “attenuate” is meant a decrease in the severity of a symptom of Fragile X Syndrome. It is understood that this is typically in relation to some standard or expected value, in other words it is relative.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, “correcting” refers to resolution of the underlying neurodegenerative disease or damage.

As used herein, “neuronal insult” means neural tissue damage produced by sudden physical injury resulting from some external condition or conditions. Nonlimiting examples of such external conditions include violence or accident, a fracture, blow, or surgical procedure.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

As used herein the term “patient” is understood to include an animal, especially a mammal, and more especially a human that is receiving or intended to receive treatment.

The term “therapeutically effective” refers to the amount of the composition used is of sufficient quantity to ameliorate one or more causes or symptoms of a disease or disorder. Such amelioration only requires a reduction or alteration, not necessarily elimination.

“Biocompatible” generally refers to a material and any metabolites or degradation products thereof that are generally non-toxic to the recipient and do not cause significant adverse effects to the subject.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years. As used herein, Tyr wild type mice refer to B6(Cg)-Tyr^{c-2J}/J, or B6-albino C57BL/6J mice that carry a mutation in the tyrosinase gene. Pigment is completely absent from skin, hair and eyes in mice homozygous for Tyr^{c-2J}.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### Methods of Treating Fragile X Syndrome (FXS)

Fragile X Syndrome (FXS) is the most common inherited form of intellectual disability and is caused by mutations in the FMR1 gene, which leads to a lack of FMRP (Fragile X Mental Retardation Protein) expression. Currently, there is no treatment for FXS. FXS has an estimated prevalence of 1 in 7,000 males and 1 in 11,000 females (Centers for Disease Control). Physical features of the disorder include mild facial anomalies, narrow face, prominent jaw, flat feet, postpubescent males and macroorchidism. Clinical symptoms of FXS can include impaired cognition, anxiety, hyperactivity, social phobia, and repetitive behaviors. FXS results from the loss of the FMR1 gene on the X chromosome. In a majority of cases, the loss is caused by an expansion of a CGG repeat region in the 5′ untranslated region of the FMR1 gene to over 230 copies, which results in hypermethylation and subsequent transcriptional inactivation. Lack of FMRP causes alterations in neurotransmitter function and intracellular signaling. Multiple targets related to FMRP and synaptic function have been identified, all supporting the hypothesis that FMRP plays an important role in synaptic plasticity. In FXS, transcripts which are typically regulated by FMRP are over translated in the dendrites and axons. FMRP regulates synaptic proteins such as Arc/Arg3.1, CamKII, MAP1B, PSD95, mTOR, MMP-9, GSK-3, Striatal-enriched protein tyrosine phosphatase (STEP), p110β and GLuR1/2 receptors. FXS appears to have excessive synaptic alpha-amino-3-hydroxy-5-methyl-4-isoxazole propionic acid (AMPA) receptor (AMPAR) internalization in response to the signaling of metabotropic glutamate receptors (mGluRs). In the absence of FMRP there is excessive mGluR-dependent protein synthesis, resulting in exaggerated mGluR-dependent long-term synaptic depression (LTD) in the hippocampus. Reducing mGluR5 signaling in FMR1 KO mice restores protein synthesis rates, hippocampal LTD and cognitive performance, suggesting that FMRP and mGluR5 act in functional opposition to maintain an optimal level of protein synthesis and thus synaptic function. Previous studies have identified changes in the levels of GluN1, GluN2A, and GluN2B proteins in FMR1 KO mice. FXS also results in a disruption of the GABAergic system with an overall dampening of the pathway, with decreases in proteins like glutamate decarboxylases (GAD) and GABA transporters (GAT). FXS is also characterized by immature dendritic spines which contribute to the dysfunction of normal synaptic plasticity. In FXS there is also a developmental delay in the conversion of silent synapses resulting in a substantial decrease in the AMPA/NMDA ratio.

Reelin protein is required for normal synaptic plasticity and its loss has been associated with several neurological diseases. Shown herein is the importance of Reelin in the regulation of many of the pathways disrupted in FXS. Disruption of expression of Reelin or its receptors results in associative and spatial learning defects, impairment of hippocampal long-term potentiation (LTP), and immature dendritic spine morphology. In humans, loss of Reelin results in a type of lissencephaly with severe cortical and cerebellar malformation.

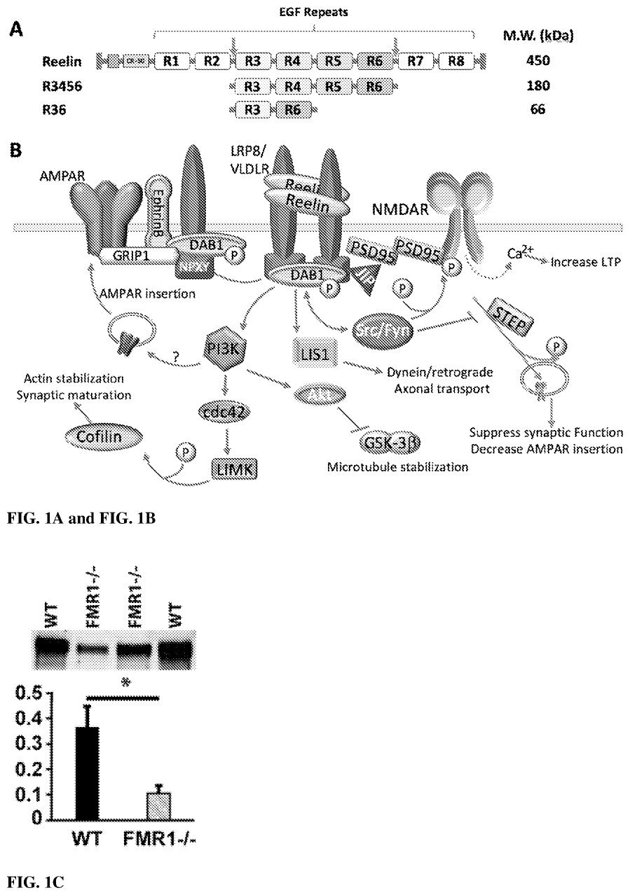

Reelin is a large ˜400 KDa extracellular signaling protein important for normal hippocampal synaptic plasticity. Reelin can be proteolytically processed in vivo at two main sites of cleavage, between repeats 2-3 and repeats 6-7 (FIG. 1A) leading to N-terminal, central, and C-terminal fragments. The central fragment, R3456 region containing REELIN repeats R3 through R6, was shown by immunoprecipitation to interact with VLDLR and LRP8 and is considered the fragment that is involved in initiating the Reelin signaling cascade. Reelin signals through two lipoprotein receptors, very-low density lipoprotein receptor (VLDLR) and low-density lipoprotein receptor-related protein 8 (LRP8 or apolipoprotein E receptor 2 (ApoER2)), causing the receptors to cluster or dimerize. Reelin binding to VLDLR and/or LRP8 leads to modulation of synaptic function and cognition by activating numerous neuronal signal transduction pathways (FIG. 1B). Receptor clustering leads to the recruitment and tyrosine phosphorylation of DAB1, via kinases from the proto-oncogene tyrosine protein family, Src and Fyn. Activation of Src and Fyn kinases leads to tyrosine phosphorylation of the NR2 subunits of NMDARs, which reduces NMDAR endocytosis and also results in increased calcium influx when those receptors are activated. Reelin supplementation potentiates glutamatergic neurotransmission, LTP, synaptic maturation and increases AMPA and NMDA receptor expression, synapse membrane localization and activity. Reelin favors the substitution of NR2B by NR2A subunits at synapses which enhances LTP. Chronic treatment with Reelin reduces the number of silent synapses in vitro. Furthermore, Reelin has been shown to increase GABAergic neurotransmission, with increases in glutamic acid decarboxylase (GAD). Reelin and LRP8 also play an important role in directing dendritic complexity through the control of actin polymerization. Briefly, Reelin signaling via PI3 kinase induces phosphorylation of LIM kinase-1 (LIMK-1), which in turn phosphorylates cofilin at an inhibitory site, thus blocking the actin-depolymerizing activity of cofilin. As a result, there is an increase in actin polymerization and dendritic spine growth with Reelin signaling and mice that overexpress Reelin have higher spine density and increased spine complexity.

Disruption of expression of Reelin or its receptors results in associative and spatial learning defects, impairment of hippocampal LTP, and immature dendritic spine morphology that ultimately impact synaptic plasticity and cognition. Reelin supplementation can transiently recover the phenotype of FMR1 KO mice (i.e., a mouse model for fragile X) from a single intracerebroventricular (ICV) REELIN protein injection. However, due to a lack of small molecule agonists for Reelin signaling, there is an absence of studies exploring effects of long-term increased Reelin signaling as a therapeutic for treating cognitive defects associated with Fragile X. The use of a protein therapy (repeated injections of Reelin protein or its fragments) has logistical issues. Therefore, a novel small active Reelin construct was generated that can be packaged into a rAAV vector for injection into the brain of Fragile X patients with the aim of achieving long-term elevated Reelin signaling.

Reelin consists of 8 EGF-like repeated domains that are proteolytically processed in vivo at two main sites of cleavage, between repeats 2-3 and repeats 6-7, with the central R3456 fragment (also referred to herein as R3-R6 or R3-6) considered critical for initiating Reelin signaling.

Recombinant Reelin fragments were formed using a full-length, human sequence of Reelin (Gene ID: 5649, Nat'l Center for Biotechnology Information, U.S. Nat'l Library of Medicine, Bethesda, Md.; Human Gene Nomenclature Committee, Cambridgeshire, UK, HGNC:9957) to determine specific regions of repeats (see U.S. Patent Application No. 2019/0169246, the content of which is incorporated by reference herein in its entirety). The fragments were commercially produced and sequenced upon arrival, prior to expression vector construction and protein production.

Full length, human REELIN cDNA nucleotide sequence
(SEQ ID NO: 1)
CACGCGTGGGCTCGGCGGGGGCCCGCTCCCAGGCCCGCTCCCGAGCCCGTTCCGCTCCCGTCCG
CCTTCTTCTCGCCTTCTCTCCGCGTGGCTCCTCCGTCCCGGCGTCTCCAAAACTGAATGAGCGA
GCGGCGCGTAGGGCGSCGGCGGCGGCGGCGGCGGCGGCGGCGGCGGCATGGAGCGCAGTGGCTG
GGCCCGGCAGACTTTCCTCCTAGCGCTGTTGCTGGGGGCGACGCTGAGGGCGCGCGCGGCGGCT
GGCTATTACCCCCGCTTTTCGCCCTTCTTTTTCCTGTGCACCCACCACGGGGAGCTGGAAGGGG
ATGGGGAGCAGGGCGAGGTGCTCATTTCCCTGCATATTGCGGGCAACCCCACCTACTACGTTCC
GGGACAAGAATACCATGTGACAATTTCAACAAGCACCTTTTTTGACGGCTTGCTGGTGACAGGA
CTATACACATCTACAAGTGTTCAGGCATCACAGAGCATTGGAGGTTCCAGTGCTTTCGGATTTG
GGATCATGTCTGACCACCAGTTTGGTAACCAGTTTATGTGCAGTGTGGTAGCCTCTCACGTGAG
TCACCTGCCCACAACCAACCTCAGTTTCATCTGGATTGCTCCACCTGCGGGCACAGGCTGTGTG
AATTTCATGGCTACAGCAACACACCGGGGCCAGGTTATTTTCAAAGATGCTTTAGCCCAGCAGT
TGTGTGAACAAGGAGCTCCAACAGATGTCACTGTGCACCCACATCTAGCTGAAATACATAGTGA
CAGCATTATCCTGAGAGATGACTTTGACTCCTACCACCAACTGCAATTAAATCCAAATATATGG
GTTGAATGTAACAACTGTGAGACTGGAGAACAGTGTGGCGCGATTATGCATGGCAATGCCGTCA
CCTTCTGTGAACCATATGGCCCACGAGAACTGATTACCACAGGCCTTAATACAACAACAGCTTC
TGTCCTCCAATTTTCCATTGGGTCAGGTTCATGTCGCTTTAGTTATTCAGACCCCAGCATCATC
GTGTTATATGCCAAGAATAACTCTGCGGACTGGATTCAGCTAGAGAAAATTAGAGCCCCTTCCA
ATGTCAGCACAATCATCCATATCCTCTACCTTCCTGAGGACGCCAAAGGGGAGAATGTCCAATT
TCAGTGGAAGCAGGAAAATCTTCGTGTAGGTGAAGTGTATGAAGCCTGCTGGGCCTTAGATAAC
ATCTTGATCATCAATTCAGCTCACAGACAAGTCGTTTTAGAAGATAGTCTCGACCCAGTGGACA
CAGGCAACTGGCTTTTCTTCCCAGGAGCTACAGTTAAGCATAGCTGTCAGTCAGATGGGAACTC
CATTTATTTCCATGGAAATGAAGGCAGCGAGTTCAATTTTGCCACCACCAGGGATGTAGATCTT
TCCACAGAAGATATTCAAGAGCAATGGTCAGAAGAATTTGAGAGCCAGCCTACAGGATGGGATG
TCTTGGGAGCTGTCATTGGTACAGAATGTGGAACGATAGAATCAGGCTTATCAATGGTCTTCCT
CAAAGATGGAGAGAGGAAATTATGCACTCCATCCATGGACACTACCGGTTATGGGAACCTGAGG
TTTTACTTTGTGATGGGAGGAATTTGTGACCCTGGAAATTCTCATGAAAATGACATAATCCTGT
ATGCAAAAATTGAAGGAAGAAAAGAGCATATAACACTGGATACCCTTTCCTATTCCTCATATAA
GGTTCCGTCTTTGGTTTCTGTGGTCATCAATCCTGAACTTCAGACTCCTGCTACCAAATTTTGT
CTCAGGCAAAAGAACCATCAAGGACATAATAGGAATGTCTGGGCTGTAGACTTTTTCCATGTCT
TGCCTGTTCTCCCTTCTACAATGTCTCACATGATACAGTTTTCCATCAATCTGGGATGTGGAAC
GCATCAGCCTGGTAACAGTGTCAGCTTGGAATTTTCTACCAACCATGGGCGCTCCTGGTCCCTC
CTTCACACTGAATGCTTACCTGAGATCTGTGCTGGACCCCACCTCCCCCACAGCACTGTCTACT
CCTCTGAAAACTACAGTGGGTGGAACCGAATAACAATTCCCCTTCCTAACGCAGCACTAACCCG
GAACACCAGGATTCGCTGGAGACAAACAGGACCAATCCTTGGAAACATGTGGGCAATTGATAAT
GTTTATATTGGCCCGTCATGTCTCAAATTCTGTTCTGGCAGAGGACAGTGCACTAGACATGGTT
GCAAGTGTGACCCTGGATTTTCTGGCCCAGCTTGTGAGATGGCATCCCAGACATTCCCAATGTT
TATTTCTGAAAGCTTTGGCAGTTCCAGGCTCTCCTCTTACCATAACTTTTACTCTATCCGTGGT
GCTGAAGTCAGCTTTGGTTGTGGTGTCTTGGCCAGTGGTAAGGCCCTGGTTTTCAACAAAGAAG
GGCGGCGTCAGCTAATTACATCTTTCCTTGACAGCTCACAATCCAGGTTTCTCCAGTTCACACT
GAGACTGGGGAGCAAATCTGTTCTGAGCACGTGCAGAGCCCCTGATCAGCCTGGTGAAGGAGTT
TTGCTGCATTATTCTTATGATAATGGGATAACTTGGAAACTCCTGGAGCATTATTCATATCTCA
GCTATCATGAGCCCAGAATAATCTCCGTAGAACTACCAGGTGATGCAAAGCAGTTTGGAATTCA
GTTCAGATGGTGGCAACCGTATCATTCTTCCCAGAGAGAAGATGTATGGGCTATTGATGAGATT
ATCATGACATCTGTGCTTTTCAACAGCATTAGTCTTGACTTTACCAATCTTGTGGAGGTCACTC
AGTCTCTGGGATTCTACCTTGGAAATGTTCAGCCATACTGTGGCCACGACTGGACCCTTTGTTT
TACAGGAGATTCTAAACTTGCCTCAAGTATGCGCTATGTGGAAACACAATCAATGCAGATAGGA
GCATCCTATATGATTCAGTTCAGTTTGGTGATGGGATGTGGCCAGAAATACACCCCACACATGG
ACAACCAGGTGAAGCTGGAGTACTCAACCAACCACGGCCTTACCTGGCACCTCGTCCAAGAAGA
ATGCCTTCCAAGTATGCCAAGTTGTCAGGAATTTACATCAGCAAGTATTTACCATGCCAGTGAG
TTTACACAGTGGAGGAGAGTCATAGTGCTTCTTCCCCAGAAAACTTGGTCCAGTGCTACCCGTT
TCCGCTGGAGCCAGAGCTATTACACAGCTCAAGACGAGTGGGCTTTGGACAGCATTTACATTGG
GCAGCAGTGCCCCAACATGTGCAGTGGGCATGGCTCATGCGATCATGGCATATGCAGGTGTGAC
CAGGGGTACCAAGGCACTGAATGCCACCCAGAAGCTGCCCTTCCGTCCACAATTATGTCAGATT
TTGAGAACCAGAATGGCTGGGAGTCTGACTGGCAAGAAGTTATTGGGGGAGAAATTGTAAAACC
AGAACAAGGGTGTGGTGTCATCTCTTCTGGATCATCTCTGTACTTCAGCAAGGCTGGGAAAAGA
CAGCTGGTGAGTTGGGACCTGGATACTTCTTGGGTGGACTTTGTCCAGTTCTACATCCAGATAG
GCGGAGAGAGTGCTTCATGCAACAAGCCTGACAGCAGAGAGGAGGGCGTCCTCCTTCAGTACAG
CAACAATGGGGGCATCCAGTGGCACCTGCTAGCAGAGATGTACTTTTCAGACTTCAGCAAACCC
AGATTTGTCTATCTGGAGCTTCCAGCTGCTGCCAAGACCCCTTGCACCAGGTTCCGCTGGTGGC
AGCCCGTGTTCTCAGGGGAGGACTATGACCAGTGGGCAGTCGATGACATCATCATTCTGTCCGA
GAAGCAGAAGCAGATCATCCCAGTTATCAATCCAACTTTACCTCAGAACTTTTATGAGAAGCCA
GCTTTTGATTACCCTATGAATCAGATGAGTGTGTGGTTGATGTTGGCTAATGAAGGAATGGTTA
AAAATGAAACCTTCTGTGCTGCCACACCATCAGCAATGATATTTGGAAAATCAGATGGAGATCG
ATTTGCAGTAACTCGAGATTTGACCCTGAAACCTGGATATGTGCTACAGTTCAAGCTAAACATA
GGTTGTGCCAATCAATTCAGCAGTACTGCTCCAGTTCTTCTTCAGTACTCTCATGATGCTGGTA
TGTCCTGGTTTCTGGTGAAAGAAGGCTGTTACCCGGCTTCTGCAGGCAAAGGATGCGAAGGAAA
CTCCAGAGAACTAAGTGAGCCCACCATGTATCACACAGGGGACTTTGAAGAATGGACAAGAATC
ACCATTGTTATTCCAAGGTCTCTTGCATCCAGCAAGACCAGATTCCGATGGATCCAGGAGAGCA
GCTCACAGAAAAACGTGCCTCCATTTGGTTTAGATGGAGTGTACATATCCGAGCCTTGTCCCAG
TTACTGCAGTGGCCATGGGGACTGCATTTCAGGAGTGTGTTTCTGTGACCTGGGATATACTGCT
GCACAAGGAACCTGTGTGTCAAATGTCCCCAATCACAATGAGATGTTCGATAGGTTTGAGGGGA
AGCTCAGCCCTCTGTGGTACAAGATAACAGGTGCCCAGGTTGGAACTGGCTGTGGAACACTTAA
CGATGGCAAATCTCTCTACTTCAATGGCCCTGGGAAAAGGGAAGCCCGGACGGTCCCTCTGGAC
ACCAGGAATATCAGACTTGTTCAATTTTATATACAAATTGGAAGCAAAACTTCAGGCATTACCT
GCATCAAACCAAGAACTAGAAATGAAGGGCTTATTGTTCAGTATTCAAATGACAATGGGATACT
CTGGCATTTGCTTCGAGAGTTGGACTTCATGTCCTTCCTGGAACCACAGATCATTTCCATTGAC
CTGCCACAGGACGCGAAGACACCTGCAACGGCATTTCGATGGTGGCAACCGCAACATGGGAAGC
ATTCAGCCCAGTGGGCTTTGGATGATGTTCTTATAGGAATGAATGACAGCTCTCAAACTGGATT
TCAAGACAAATTTGATGGCTCTATAGATTTGCAAGCCAACTGGTATCGAATCCAAGGAGGTCAA
GTTGATATTGACTGTCTCTCTATGGATACTGCTCTGATATTCACTGAAAACATAGGAAAACCTC
GTTATGCTGAGACCTGGGATTTTCATGTGTCAGCATCTACCTTTTTGCAGTTTGAAATGAGCAT
GGGCTGTAGCAAGCCCTTCAGCAACTCCCACAGTGTACAGCTCCAGTATTCTCTGAACAATGGC
AAGGACTGGCATCTTGTCACCGAAGAGTGTGTTCCTCCAACCATTGGCTGTCTGCATTACACGG
AAAGTTCAATTTACACCTCGGAAAGATTCCAGAATTGGAAGCGGATCACTGTCTACCTTCCACT
CTCCACCATTTCTCCCAGGACCCGGTTCAGATGGATTCAGGCCAACTACACTGTGGGGGCTGAT
TCCTGGGCGATTGATAATGTTGTACTGGCCTCAGGGTGCCCTTGGATGTGCTCAGGACGAGGGA
TTTGTGATGCTGGACGCTGTGTGTGTGACCGGGGCTTTGGTGGACCCTATTGTGTTCCTGTTGT
TCCTCTGCCCTCGATTCTTAAAGACGATTTCAATGGGAATTTACATCCTGACCTTTGGCCTGAA
GTGTATGGTGCAGAGAGGGGGAATCTGAATGGTGAAACCATCAAATCTGGAACATCTCTAATTT
TTAAAGGGGAAGGACTAAGGATGCTTATTTCAAGAGATCTAGATTGTACAAATACAATGTATGT
CCAGTTTTCACTTAGATTTATAGCAAAAAGTACCCCAGAGAGATCTCACTCTATTCTGTTACAA
TTCTCCATCAGTGGAGGAATCACTTGGCACCTGATGGATGAATTTTACTTTCCTCAAACAACGA
ATATACTTTTCATCAATGTTCCCTTGCCATACACTGCCCAAACCAATGCTACAAGATTCAGACT
CTGGCAACCTTATAATAACGGTAAGAAAGAAGAAATCTGGATTGTTGATGACTTCATTATCGAT
GGAAATAATGTAAACAACCCTGTGATGCTCTTGGATACATTTGATTTTGGGCCCAGAGAAGACA
ATTGGTTTTTCTATCCTGGTGGTAACATCGGTCTTTATTGTCCATATTCTTCAAAGGGGGCACC
TGAAGAAGATTCAGCTATGGTGTTTGTTTCAAATGAAGTTGGTGAGCATTCCATTACCACCCGT
GACCTAAATGTGAATGAGAACACCATCATACAATTTGAGATCAACGTTGGCTGTTCGACTGATA
GCTCATCCGCGGATCCAGTGAGACTGGAATTTTCAAGGGACTTCGGGGCGACCTGGCACCTTCT
GCTGCCCCTCTGCTACCACAGCAGCAGCCACGTCAGCTCTTTATGCTCCACCGAGCACCACCCC
AGCAGCACCTACTACGCAGGAACCATGCAGGGCTGGAGGAGGGAGGTCGTGCACTTTGGGAAGC
TGCACCTTTGTGGATCTGTCCGTTTCAGATGGTACCAGGGATTTTACCCTGCCGGCTCTCAGCC
AGTGACATGGGCCATTGATAATGTCTACATCGGTCCCCAGTGTGAGGAGATGTGTAATGGACAG
GGGAGCTGTATCAATGGAACCAAATGTATATGTGACCCTGGCTACTCAGGTCCAACCTGTAAAA
TAAGCACCAAAAATCCTGATTTTCTCAAAGATGATTTCGAAGGTCAGCTAGAATCTGATAGATT
CTTATTAATGAGTGGTGGGAAACCATCTCGAAAGTGTGGAATCCTTTCTAGTGGAAACAACCTC
TTTTTCAATGAAGATGGCTTGCGCATGTTGATGACACGAGACCTGGATTTATCACATGCTAGAT
TTGTGCAGTTCTTCATGAGACTGGGATGTGGTAAAGGCGTTCCTGACCCCAGGAGTCAACCCGT
GCTCCTACAGTATTCTCTCAACGGTGGCCTCTCGTGGAGTCTTCTTCAGGAGTTCCTTTTCAGC
AATTCCAGCAATGTGGGCAGGTACATTGCCCTGGAGATACCCTTGAAAGCCCGTTCTGGTTCTA
CTCGCCTTCGCTGGTGGCAACCGTCTGAGAATGGGCACTTCTACAGCCCCTGGGTTATCGATCA
GATTCTTATTGGAGGAAATATTTCTGGTAATACGGTCTTGGAAGATGATTTCACAACCCTTGAT
AGTAGGAAATGGCTGCTTCACCCAGGAGGCACCAAGATGCCCGTGTGTGGCTCTACTGGTGATG
CCCTGGTCTTCATTGAAAAGGCCAGCACCCGTTACGTGGTCAGCACAGACGTTGCCGTGAATGA
GGATTCCTTCCTACAGATAGACTTCGCTGCCTCCTGCTCAGTCACAGACTCTTGTTATGCGATT
GAATTGGAATACTCAGTAGATCTTGGATTGTCATGGCACCCATTGGTAAGGGACTGTCTGCCTA
CCAATGTGGAATGCAGTCGCTATCATCTGCAACGGATCCTGGTGTCAGACACTTTCAACAAGTG
GACTAGAATCACTCTGCCTCTCCCTCCTTATACCAGGTCCCAAGCCACTCGTTTCCGTTGGCAT
CAACCAGCTCCTTTTGACAAGCAGCAGACATGGGCAATAGATAATGTCTATATCGGGGATGGCT
GCATAGACATGTGCAGTGGCCATGGGAGATGCATCCAGGGAAACTGCGTCTGTGATGAACAGTG
GGGTGGCCTGTACTGTGATGACCCCGAGACCTCTCTTCCAACCCAACTCAAAGACAACTTCAAT
CGAGCTCCATCCAGTCAGAACTGGCTGACTGTGAACGGAGGGAAATTGAGTACAGTGTGTGGAG
CCGTGGCGTCGGGAATGGCTCTCCATTTCAGTGGGGGTTGTAGTCGATTATTAGTCACTGTGGA
TCTAAACCTCACTAATGCTGAGTTCATCCAATTTTACTTCATGTATGGGTGCCTGATTACACCA
AACAACCGTAACCAAGGTGTTCTCTTGGAATATTCTGTCAATGGAGGCATTACCTGGAACCTGC
TCATGGAGATTTTCTATGACCAGTACAGTAAGCCCGGATTTGTGAATATCCTTCTCCCTCCTGA
TGCTAAAGAGATTGCCACTCGCTTCCGCTGGTGGCAGCCAAGACATGACGGCCTGGATCAGAAC
GACTGGGCCATTGACAATGTCCTCATCTCAGGCTCTGCTGACCAAAGGACCGTTATGCTGGACA
CCTTCAGCAGCGCCCCAGTACCCCAGCACGAGCGCTCCCCTGCAGATGCCGGCCCTGTCGGGAG
GATCGCCTTTGACATGTTTATGGAAGACAAAACTTCAGTGAATGAGCACTGGCTATTCCATGAT
GATTGTACAGTAGAAAGATTCTGTGACTCCCCTGATGGTGTGATGCTCTGTGGCAGTCATGATG
GACGGGAGGTGTATGCAGTGACCCATGACCTGACTCCCACTGAAGGCTGGATTATGCAATTCAA
GATCTCAGTTGGATGTAAGGTGTCTGAAAAAATTGCCCAGAATCAAATTCATGTGCAGTATTCT
ACTGACTTCGGTGTGAGTTGGAATTATCTGGTCCCTCAGTGCTTGCCTGCTGACCCAAAATGCT
CTGGAAGTGTTTCTCAGCCATCTGTATTCTTTCCAACTAAAGGGTGGAAAAGGATCACCTACCC
ACTTCCTGAAAGCTTAGTGGGAAATCCGGTAAGGTTTAGGTTCTATCAGAAGTACTCAGACATG
CAGTGGGCAATCGATAATTTCTACCTGGGCCCTGGATGCTTGGACAACTGCAGGGGCCATGGAG
ATTGCTTAAGGGAACAGTGCATCTGTGATCCGGGATACTCAGGGCCAAACTGCTACTTGACCCA
CACTCTGAAGACTTTCCTGAAGGAACGCTTTGACAGTGAAGAAATCAAACCTGACTTATGGATG
TCCTTAGAAGGTGGAAGTACTTGCACTGAGTGTGGAATTCTTGCCGAGGACACTGCACTCTATT
TTGGGGGATCCACTGTGAGACAAGCGGTTACACAAGATTTGGATCTTCGAGGTGCAAAGTTCCT
GCAATACTGGGGGCGCATCGGTAGTGAGAACAACATGACCTCTTGCCATCGTCCCATCTGCCGG
AAGGAAGGCGTGCTGTTGGACTACTCTACCGATGGAGGAATTACCTGGACTTTGCTCCATGAGA
TGGATTACCAGAAATACATTTCTGTTAGACACGACTACATACTTCTTCCTGAAGATGCCCTCAC
CAACACAACTCGACTTCGCTGGTGGCAGCCTTTTGTGATCAGCAATGGAATTGTGGTCTCTGGG
GTGGAGCGTGCTCAGTGGGCACTGGACAACATTTTGATTGGTGGAGCAGAAATCAATCCCAGCC
AATTGGTGGACACTTTTGATGATGAAGGCACTTCCCATGAAGAAAACTGGAGTTTTTACCCTAA
TGCTGTAAGGACAGCAGGATTTTGTGGCAATCCATCCTTTCACCTCTATTGGCCAAATAAAAAG
AAGGACAAGACTCACAATGCTCTCTCCTCCCGAGAACTCATTATACAGCCAGGATACATGATGC
AGTTTAAAATTGTGGTGGGTTGTGAAGCCACTTCTTGTGGTGACCTTCATTCCGTAATGCTGGA
ATACACTAAGGATGCAAGATCGGATTCCTGGCAGCTCGTACAGACCCAGTGCCTTCCTTCCTCT
TCTAACAGCATTGGCTGCTCCCCTTTCCAGTTCCATGAAGCCACCATCTACAACTCTGTCAACA
GCTCAAGCTGGAAAAGAATCACCATCCAGCTGCCTGACCATGTCTCCTCTAGTGCAACACAGTT
CCGCTGGATCCAGAAGGGAGAAGAAACTGAGAAGCAAAGCTGGGCAATTGACCACGTGTACATT
GGAGAGGCTTGCCCCAAGCTCTGCAGCGGGCACGGATACTGCACGACCGGTGCCATCTGCATCT
GCGACGAGAGCTTCCAAGGTGATGACTGCTCTGTTTTCAGTCACGACCTTCCCAGTTATATTAA
AGATAATTTTGAGTCCGCAAGAGTCACCGAGGCAAACTGGGAGACCATTCAAGGTGGAGTCATA
GGAAGTGGCTGTGGGCAGCTGGCCCCCTACGCCCATGGAGACTCACTGTACTTTAATGGCTGTC
AGATCAGGCAAGCAGCTACCAAGCCTCTGGATCTCACTCGAGCAAGCAAAATCATGTTTGTTTT
GCAAATTGGGAGCATGTCGCAGACGGACAGCTGCAACAGTGACCTGAGTGGCCCCCACGCTGTG
GACAAGGCGGTGCTGCTGCAATACAGCGTCAACAACGGGATCACCTGGCATGTCATCGCCCAGC
ACCAGCCAAAGGACTTCACACAAGCTCAGAGAGTGTCTTACAATGTCCCCCTGGAGGCACGGAT
GAAAGGAGTCTTACTGCGCTGGTGGCAACCACGCCACAATGGAACAGGTCATGATCAATGGGCT
TTGGACCATGTGGAGGTCGTCCTAGTAAGCACTCGCAAACAAAATTACATGATGAATTTTTCAC
GACAACATGGGCTCAGACATTTCTACAACAGAAGACGAAGGTCACTTAGGCGATACCCATGAAG
AATCAAAAAGTTTATTTTTTTTCTTCCAACATGTGATGTGTTGCTCTCCATTCTTTTAAATCTC
GCACTACATCTGATATCAGGAAATATCTGTGAAGGACTTGGTGATTACCTGAAAGCCCTTCTCA
AGACCGAGTGTACACCACTTTCCCACACTGTGAACTAATGACAAGTGACTTATTTGCTCATAAG
TAAATGTCTTCATGTTGATGTGTCCGTGAAAGTTGTGATCTGTTGTAATATCAGTTACAGTGGC
AGTATTGACAATAAGAAACAGTTTAACAGAAAAATGAAATTTAAGCACAAAAAATTTAAGAGAT
TTTATGTTTAAAATGGCATTTAGCACAGTATTTAACATTCTTGGTCACAAAGCTATTTAAGTGG
ACTGTATTTCAGCTATGTCTCATGTTTTATATGATTAAATTATCATTGTTTGTCCTTTATGTAT
TCTCTTCTACAATACAACACATTGAAACTGTATTTACTTGTTATGTTGTAATATTTTGCTGCTG
AATTTGGGGCTACTTATATTCTGCAGAAAATTAATTGAAATACCTATTCAAGAAGATAGTTGTA
AAGATATTGTATCTCCTTTAATATACTCCTTAAAAATGTATGTTGGTTTAGCGTTGTTTTGTGG
ATAAGAAAAATGCTTGACCCTGAAATATTTTCTACTTTAAATTGTGGATGAAGACCCTATCTCC
CACAAATAAGTTCCCATTTCCTTGTCTAAAGATCTTTTTTTAAGTGTTCTGTGGCTGATTTACT
AACAGTAACTGCCATTTTTTGTCTGTGATAACAGAGTGATTTGTAAAACAGTGGTTGTTTTTTC
ATTGTGTTTTCTTCGTGGATTGTTTTTTCTGCGGGTCATATTCATACCTTCTGATGAAGTTGTA
CAACACCAGCAACATTATAATGGCCCTGTAGCTCTGAATGCTATTTGTGTAACTGAAAGGTTGC
ACTCTAGGGTGAACCAAGCTATAAAAGCCCATGCTTAAATAAAAATTATGTCCAAAAGCC

HEK293 cells were stably transfected with the full-length Reelin gene in a pCrl vector for the production of REELIN protein fragments. A full length Reelin expression vector was transfected into the HEK293 cells, as previously described (Weeber, et al., Reelin and ApoE receptors cooperate to enhance hippocampal synaptic plasticity and learning. J. Biol. Chem. 2002; 277:39944-39952; Sinagra, et al., Reelin, very-low-density lipoprotein receptor, and apolipoprotein E receptor 2 control somatic NMDA receptor composition during hippocampal maturation in vitro. J. Neurosci. 2005; 25:6127-6136). Once confluent, the cells were grown in low-glucose Dulbecco's modified Eagle's medium with 0.2% bovine serum albumin for 2 days, followed by media collection, sterile filtration, and concentration by Centricon Plus-80 centrifugal filter units (Millipore). REELIN protein was cleaved extracellularly at two sites, resulting in the generation of three major fragments: the N-terminus to repeat 2 (roughly 180 kDa), the central fragment from repeat 3-6 (roughly 190 kDa), and the C-terminal fragment consisting of repeats 7 and 8 (roughly 80 kDa) (Nakajima, et al., Disruption of hippocampal development in vivo by CR-50 mAb against reelin. Proc. Natl. Acad. Sci. USA. 1997; 94: 8196-820; de Rouvroit, et al., (1999) REELIN, the extracellular matrix protein deficient in reeler mutant mice, is processed by a metalloproteinase. Exp. Neurol. 1999; 156:214-217; Utsunomiya-Tate, et al., Reelin molecules assemble together to form a large protein complex, which is inhibited by the function-blocking CR-50 antibody. Proc. Natl. Acad. Sci. USA. 2000; 97: 9729-9734; Jossin, et al., The central fragment of Reelin, generated by proteolytic processing in vivo, is critical to its function during cortical plate development. J. Neurosci. 2004; 24:514-521; Jossin, et al., Processing of Reelin by embryonic neurons is important for function in tissue but not in dissociated cultured neurons. J. Neurosci. 2007; 27:4243-4252; Koie, et al., Cleavage within Reelin repeat 3 regulates the duration and range of the signaling activity of Reelin protein. J. Biol. Chem. 2014; 289:12922-12930; Krstic, et al., Regulated proteolytic processing of Reelin through interplay of tissue plasminogen activator (tPA), ADAMTS-4, ADAMTS-5, and their modulators. PLoS One. 2012; 7:e47793; Trotter, et al., Extracellular proteolysis of reelin by tissue plasminogen activator following synaptic potentiation. Neuroscience. 2014; 274:299-307). Additionally, two intermediate fragments were produced: one consisting of the N-terminus to repeat 6 (roughly 370 kDa), and one consisting of repeats 6-8 (roughly 270 kDa) (Jossin, et al., The central fragment of Reelin, generated by proteolytic processing in vivo, is critical to its function during cortical plate development. J. Neurosci. 2004; 24:514-521).

For the generation of recombinant REELIN, two or more REELIN DNA fragments encoding specific REELIN repeat protein fragments were ligated into an expression vector or viral vector using standard procedures. The REELIN repeat constructs were then sequence verified prior to being. transfected into HEK293 cells.

As used herein the phrase “construct formed from fragment repeats of Reelin” refers to an artificial protein generated from fragments obtained from combining repeat regions of Reelin. As seen in the specification, full-length Reelin is comprised of regions of DNA or amino acids (for the protein) that are termed repeats, such as regions R1, R2, R3, R4, R5, R6, R7, and R8. Loop regions are located between these repeat regions, which are used in joining two repeat regions.

As used herein “loop region” means a section of a Reelin nucleic acid sequence that corresponds to an RNA loop structure, and which is disposed between two repeat regions, and joins the two repeat regions. The term “repeat region” means a section of a Reelin nucleic acid sequence that forms a fundamental recurring unit. Specific “repeat regions” are disclosed throughout the specification. In specific embodiments, the “loop region” is a structure formed by a single strand of nucleic acid having complementary regions that flank a particular single stranded nucleotide region hybridize in a way that the single stranded nucleotide region between the complementary regions is excluded from duplex formation or Watson-Crick base pairing.

REELIN Repeat 3 (R3) nucleotide sequence.
(SEQ ID NO: 2)
TTCAGCAGTACTGCTCCAGTTCTTCTTCAGTACTCTCATGATGCTGGTATGTCCTGGTT
TCTGGTGAAAGAAGGCTGTTACCCGGCTTCTGCAGGCAAAGGATGCGAAGGAAACTCCAGAGAA
CTAAGTGAGCCCACCATGTATCACACAGGGGACTTTGAAGAATGGACAAGAATCACCATTGTTA
TTCCAAGGTCTCTTGCATCCAGCAAGACCAGATTCCGATGGATCCAGGAGAGCAGCTCACAGAA
AAACGTGCCTCCATTTGGTTTAGATGGAGTGTACATATCCGAGCCTTGTCCCAGTTACTGCAGT
GGCCATGGGGACTGCATTTCAGGAGTGTGTTTCTGTGACCTGGGATATACTGCTGCACAAGGAA
CCTGTGTGTCAAATGTCCCCAATCACAATGAGATGTTCGATAGGTTTGAGGGGAAGCTCAGCCC
TCTGTGGTACAAGATAACAGGTGCCCAGGTTGGAACTGGCTGTGGAACACTTAACGATGGCAAA
TCTCTCTACTTCAATGGCCCTGGGAAAAGGGAAGCCCGGACGGTCCCTCTGGACACCAGGAATA
TCAGACTTGTTCAATTTTATATACAAATTGGAAGCAAAACTTCAGGCATTACCTGCATCAAACC
AAGAACTAGAAATGAAGGGCTTATTGTTCAGTATTCAAATGACAATGGGATACTCTGGCATTTG
CTTCGAGAGTTGGACTTCATGTCCTTCCTG
REELIN Repeat 4 (R4) nucleotide sequence.
(SEQ ID NO: 3)
CCCTTCAGCAACTCCCACAGTGTACAGCTCCAGTATTCTCTGAACAATGGCAAGGACTG
GCATCTTGTCACCGAAGAGTGTGTTCCTCCAACCATTGGCTGTCTGCATTACACGGAAAGTTCA
ATTTACACCTCGGAAAGATTCCAGAATTGGAAGCGGATCACTGTCTACCTTCCACTCTCCACCA
TTTCTCCCAGGACCCGGTTCAGATGGATTCAGGCCAACTACACTGTGGGGGCTGATTCCTGGGC
GATTGATAATGTTGTACTGGCCTCAGGGTGCCCTTGGATGTGCTCAGGACGAGGGATTTGTGAT
GCTGGACGCTGTGTGTGTGACCGGGGCTTTGGTGGACCCTATTGTGTTCCTGTTGTTCCTCTGC
CCTCGATTCTTAAAGACGATTTCAATGGGAATTTACATCCTGACCTTTGGCCTGAAGTGTATGG
TGCAGAGAGGGGGAATCTGAATGGTGAAACCATCAAATCTGGAACATCTCTAATTTTTAAAGGG
GAAGGACTAAGGATGCTTATTTCAAGAGATCTAGATTGTACAAATACAATGTATGTCCAGTTTT
CACTTAGATTTATAGCAAAAAGTACCCCAGAGAGATCTCACTCTATTCTGTTACAATTCTCCAT
CAGTGGAGGAATCACTTGGCACCTGATGGATGAATTTTACTTTCCTCAAACAACG
REELIN Repeat 5 (R5) nucleotide sequence.
(SEQ ID NO: 4)
GATAGCTCATCCGCGGATCCAGTGAGACTGGAATTTTCAAGGGACTTCGGGGCGACCTG
GCACCTTCTGCTGCCCCTCTGCTACCACAGCAGCAGCCACGTCAGCTCTTTATGCTCCACCGAG
CACCACCCCAGCAGCACCTACTACGCAGGAACCATGCAGGGCTGGAGGAGGGAGGTCGTGCACT
TTGGGAAGCTGCACCTTTGTGGATCTGTCCGTTTCAGATGGTACCAGGGATTTTACCCTGCCGG
CTCTCAGCCAGTGACATGGGCCATTGATAATGTCTACATCGGTCCCCAGTGTGAGGAGATGTGT
AATGGACAGGGGAGCTGTATCAATGGAACCAAATGTATATGTGACCCTGGCTACTCAGGTCCAA
CCTGTAAAATAAGCACCAAAAATCCTGATTTTCTCAAAGATGATTTCGAAGGTCAGCTAGAATC
TGATAGATTCTTATTAATGAGTGGTGGGAAACCATCTCGAAAGTGTGGAATCCTTTCTAGTGGA
AACAACCTCTTTTTCAATGAAGATGGCTTGCGCATGTTGATGACACGAGACCTGGATTTATCAC
ATGCTAGATTTGTGCAGTTCTTCATGAGACTGGGATGTGGTAAAGGCGTTCCTGACCCCAGGAG
TCAACCCGTGCTCCTACAGTATTCTCTCAACGGTGGCCTCTCGTGGAGTCTTCTTCAGGAGTTC
CTTTTCAGCAATTCCAGC
REELIN Repeat 6 (R6) nucleotide sequence.
(SEQ ID NO: 5)
GTCACAGACTCTTGTTATGCGATTGAATTGGAATACTCAGTAGATCTTGGATTGTCATG
GCACCCATTGGTAAGGGACTGTCTGCCTACCAATGTGGAATGCAGTCGCTATCATCTGCAACGG
ATCCTGGTGTCAGACACTTTCAACAAGTGGACTAGAATCACTCTGCCTCTCCCTCCTTATACCA
GGTCCCAAGCCACTCGTTTCCGTTGGCATCAACCAGCTCCTTTTGACAAGCAGCAGACATGGGC
AATAGATAATGTCTATATCGGGGATGGCTGCATAGACATGTGCAGTGGCCATGGGAGATGCATC
CAGGGAAACTGCGTCTGTGATGAACAGTGGGGTGGCCTGTACTGTGATGACCCCGAGACCTCTC
TTCCAACCCAACTCAAAGACAACTTCAATCGAGCTCCATCCAGTCAGAACTGGCTGACTGTGAA
CGGAGGGAAATTGAGTACAGTGTGTGGAGCCGTGGCGTCGGGAATGGCTCTCCATTTCAGTGGG
GGTTGTAGTCGATTATTAGTCACTGTGGATCTAAACCTCACTAATGCTGAGTTCATCCAATTTT
ACTTCATGTATGGGTGCCTGATTACACCAAACAACCGTAACCAAGGTGTTCTCTTGGAATATTC
TGTCAATGGAGGCATTACCTGGAACCTGCTCATGGAGATTTTCTATGACCAGTACAGT
REELIN Repeat Loop Region 3-4 nucleotide sequence.
(SEQ ID NO: 6)
GAACCACAGATCATTTCCATTGACCTGCCACAGGACGCGAAGACACCTGCAACGGCATT
TCGATGGTGGCAACCGCAACATGGGAAGCATTCAGCCCAGTGGGCTTTGGATGATGTTCTTATA
GGAATGAATGACAGCTCTCAAACTGGATTTCAAGACAAATTTGATGGCTCTATAGATTTGCAAG
CCAACTGGTATCGAATCCAAGGAGGTCAAGTTGATATTGACTGTCTCTCTATGGATACTGCTCT
GATATTCACTGAAAACATAGGAAAACCTCGTTATGCTGAGACCTGGGATTTTCATGTGTCAGCA
TCTACCTTTTTGCAGTTTGAAATGAGCATGGGCTGTAGCAAG
REELIN Repeat Loop Region 4-5 nucleotide sequence.
(SEQ ID NO: 7)
AATATACTTTTCATCAATGTTCCCTTGCCATACACTGCCCAAACCAATGCTACAAGATT
CAGACTCTGGCAACCTTATAATAACGGTAAGAAAGAAGAAATCTGGATTGTTGATGACTTCATT
ATCGATGGAAATAATGTAAACAACCCTGTGATGCTCTTGGATACATTTGATTTTGGGCCCAGAG
AAGACAATTGGTTTTTCTATCCTGGTGGTAACATCGGTCTTTATTGTCCATATTCTTCAAAGGG
GGCACCTGAAGAAGATTCAGCTATGGTGTTTGTTTCAAATGAAGTTGGTGAGCATTCCATTACC
ACCCGTGACCTAAATGTGAATGAGAACACCATCATACAATTTGAGATCAACGTTGGCTGTTCGA
CT
REELIN Repeat Loop Region 5-6 nucleotide sequence.
(SEQ ID NO: 8)
AATGTGGGCAGGTACATTGCCCTGGAGATACCCTTGAAAGCCCGTTCTGGTTCTACTCG
CCTTCGCTGGTGGCAACCGTCTGAGAATGGGCACTTCTACAGCCCCTGGGTTATCGATCAGATT
CTTATTGGAGGAAATATTTCTGGTAATACGGTCTTGGAAGATGATTTCACAACCCTTGATAGTA
GGAAATGGCTGCTTCACCCAGGAGGCACCAAGATGCCCGTGTGTGGCTCTACTGGTGATGCCCT
GGTCTTCATTGAAAAGGCCAGCACCCGTTACGTGGTCAGCACAGACGTTGCCGTGAATGAGGAT
TCCTTCCTACAGATAGACTTCGCTGCCTCCTGCTCA
Recombinant human R3 + R6 or R36 REELIN nucleotide sequence, R3
fragment conjugated to the R6 fragment.
(SEQ ID NO: 9)
AAGCTTCCACCATGGAGCGCAGTGGCTGGGCCCGGCAGACTTTCCTCCTAGCGCTGTTG
CTGGGGGCGACGCTGAGGGCGCGCGCGTTCAGCAGTACTGCTCCAGTTCTTCTTCAGTACTCTC
ATGATGCTGGTATGTCCTGGTTTCTGGTGAAAGAAGGCTGTTACCCGGCTTCTGCAGGCAAAGG
ATGCGAAGGAAACTCCAGAGAACTAAGTGAGCCCACCATGTATCACACAGGGGACTTTGAAGAA
TGGACAAGAATCACCATTGTTATTCCAAGGTCTCTTGCATCCAGCAAGACCAGATTCCGATGGA
TCCAGGAGAGCAGCTCACAGAAAAACGTGCCTCCATTTGGTTTAGATGGAGTGTACATATCCGA
GCCTTGTCCCAGTTACTGCAGTGGCCATGGGGACTGCATTTCAGGAGTGTGTTTCTGTGACCTG
GGATATACTGCTGCACAAGGAACCTGTGTGTCAAATGTCCCCAATCACAATGAGATGTTCGATA
GGTTTGAGGGGAAGCTCAGCCCTCTGTGGTACAAGATAACAGGTGCCCAGGTTGGAACTGGCTG
TGGAACACTTAACGATGGCAAATCTCTCTACTTCAATGGCCCTGGGAAAAGGGAAGCCCGGACG
GTCCCTCTGGACACCAGGAATATCAGACTTGTTCAATTTTATATACAAATTGGAAGCAAAACTT
CAGGCATTACCTGCATCAAACCAAGAACTAGAAATGAAGGGCTTATTGTTCAGTATTCAAATGA
CAATGGGATACTCTGGCATTTGCTTCGAGAGTTGGACTTCATGTCCTTCCTGGAACCACAGATC
ATTTCCATTGACCTGCCACAGGACGCGAAGACACCTGCAACGGCATTTCGATGGTGGCAACCGC
AACATGGGAAGCATTCAGCCCAGTGGGCTTTGGATGATGTTCTTATAGGAATGAATGACAGCTC
TCAAACTGGATTTCAAGACAAATTTGATGGCTCTATAACCCTTGATAGTAGGAAATGGCTGCTT
CACCCAGGAGGCACCAAGATGCCCGTGTGTGGCTCTACTGGTGATGCCCTGGTCTTCATTGAAA
AGGCCAGCACCCGTTACGTGGTCAGCACAGACGTTGCCGTGAATGAGGATTCCTTCCTACAGAT
AGACTTCGCTGCCTCCTGCTCAGTCACAGACTCTTGTTATGCGATTGAATTGGAATACTCAGTA
GATCTTGGATTGTCATGGCACCCATTGGTAAGGGACTGTCTGCCTACCAATGTGGAATGCAGTC
GCTATCATCTGCAACGGATCCTGGTGTCAGACACTTTCAACAAGTGGACTAGAATCACTCTGCC
TCTCCCTCCTTATACCAGGTCCCAAGCCACTCGTTTCCGTTGGCATCAACCAGCTCCTTTTGAC
AAGCAGCAGACATGGGCAATAGATAATGTCTATATCGGGGATGGCTGCATAGACATGTGCAGTG
GCCATGGGAGATGCATCCAGGGAAACTGCGTCTGTGATGAACAGTGGGGTGGCCTGTACTGTGA
TGACCCCGAGACCTCTCTTCCAACCCAACTCAAAGACAACTTCAATCGAGCTCCATCCAGTCAG
AACTGGCTGACTGTGAACGGAGGGAAATTGAGTACAGTGTGTGGAGCCGTGGCGTCGGGAATGG
CTCTCCATTTCAGTGGGGGTTGTAGTCGATTATTAGTCACTGTGGATCTAAACCTCACTAATGC
TGAGTTCATCCAATTTTACTTCATGTATGGGTGCCTGATTACACCAAACAACCGTAACCAAGGT
GTTCTCTTGGAATATTCTGTCAATGGAGGCATTACCTGGAACCTGCTCATGGAGATTTTCTATG
ACCAGTACAGTGATTACAAGGATGACGACGATAAGTGACTCGAG
Amino acid sequence of recombinant human R3 + R6 or R36 REELIN
protein fragment, R3 fragment conjugated to the R6 fragment,
i.e. Reelin protein fragment.
(SEQ ID NO: 10)
MERSGWARQTFLLALLLGATLRARAFSSTAPVLLQYSHDAGMSWFLVKEGCYPASAGKG
CEGNSRELSEPTMYHTGDFEEWTRITIVIPRSLASSKTRFRWIQESSSQKNVPPFGLDGVYISE
PCPSYCSGHGDCISGVCFCDLGYTAAQGTCVSNVPNHNEMFDRFEGKLSPLWYKITGAQVGTGC
GTLNDGKSLYFNGPGKREARTVPLDTRNIRLVQFYIQIGSKTSGITCIKPRTRNEGLIVQYSND
NGILWHLLRELDFMSFLEPQIISIDLPQDAKTPATAFRWWQPQHGKHSAQWALDDVLIGMNDSS
QTGFQDKFDGSITLDSRKWLLHPGGTKMPVCGSTGDALVFIEKASTRYVVSTDVAVNEDSFLQI
DFAASCSVTDSCYAIELEYSVDLGLSWHPLVRDCLPTNVECSRYHLQRILVSDTFNKWTRITLP
LPPYTRSQATRFRWHQPAPFDKQQTWAIDNVYIGDGCIDMCSGHGRCIQGNCVCDEQWGGLYCD
DPETSLPTQLKDNFNRAPSSQNWLTVNGGKLSTVCGAVASGMALHFSGGCSRLLVTVDLNLTNA
EFIQFYFMYGCLITPNNRNQGVLLEYSVNGGITWNLLMEIFYDQYS
Recombinant human R3 + R5 or R35 REELIN gene nucleotide sequence,
R3 fragment conjugated to the R5 fragment.
(SEQ ID NO: 11)
AAGCTTCCACCATGGAGCGCAGTGGCTGGGCCCGGCAGACTTTCCTCCTAGCGCTGTTG
CTGGGGGCGACGCTGAGGGCGCGCGCGTTCAGCAGTACTGCTCCAGTTCTTCTTCAGTACTCTC
ATGATGCTGGTATGTCCTGGTTTCTGGTGAAAGAAGGCTGTTACCCGGCTTCTGCAGGCAAAGG
ATGCGAAGGAAACTCCAGAGAACTAAGTGAGCCCACCATGTATCACACAGGGGACTTTGAAGAA
TGGACAAGAATCACCATTGTTATTCCAAGGTCTCTTGCATCCAGCAAGACCAGATTCCGATGGA
TCCAGGAGAGCAGCTCACAGAAAAACGTGCCTCCATTTGGTTTAGATGGAGTGTACATATCCGA
GCCTTGTCCCAGTTACTGCAGTGGCCATGGGGACTGCATTTCAGGAGTGTGTTTCTGTGACCTG
GGATATACTGCTGCACAAGGAACCTGTGTGTCAAATGTCCCCAATCACAATGAGATGTTCGATA
GGTTTGAGGGGAAGCTCAGCCCTCTGTGGTACAAGATAACAGGTGCCCAGGTTGGAACTGGCTG
TGGAACACTTAACGATGGCAAATCTCTCTACTTCAATGGCCCTGGGAAAAGGGAAGCCCGGACG
GTCCCTCTGGACACCAGGAATATCAGACTTGTTCAATTTTATATACAAATTGGAAGCAAAACTT
CAGGCATTACCTGCATCAAACCAAGAACTAGAAATGAAGGGCTTATTGTTCAGTATTCAAATGA
CAATGGGATACTCTGGCATTTGCTTCGAGAGTTGGACTTCATGTCCTTCCTGGAACCACAGATC
ATTTCCATTGACCTGCCACAGGACGCGAAGACACCTGCAACGGCATTTCGATGGTGGCAACCGC
AACATGGGAAGCATTCAGCCCAGTGGGCTTTGGATGATGTTCTTATAGGAATGAATGACAGCTC
TCAAACTGGATTTCAAGACAAATTTGATGGCTCTATAGATGACAATTGGTTTTTCTATCCTGGT
GGTAACATCGGTCTTTATTGTCCATATTCTTCAAAGGGGGCACCTGAAGAAGATTCAGCTATGG
TGTTTGTTTCAAATGAAGTTGGTGAGCATTCCATTACCACCCGTGACCTAAATGTGAATGAGAA
CACCATCATACAATTTGAGATCAACGTTGGCTGTTCGACTGATAGCTCATCCGCGGATCCAGTG
AGACTGGAATTTTCAAGGGACTTCGGGGCGACCTGGCACCTTCTGCTGCCCCTCTGCTACCACA
GCAGCAGCCACGTCAGCTCTTTATGCTCCACCGAGCACCACCCCAGCAGCACCTACTACGCAGG
AACCATGCAGGGCTGGAGGAGGGAGGTCGTGCACTTTGGGAAGCTGCACCTTTGTGGATCTGTC
CGTTTCAGATGGTACCAGGGATTTTACCCTGCCGGCTCTCAGCCAGTGACATGGGCCATTGATA
ATGTCTACATCGGTCCCCAGTGTGAGGAGATGTGTAATGGACAGGGGAGCTGTATCAATGGAAC
CAAATGTATATGTGACCCTGGCTACTCAGGTCCAACCTGTAAAATAAGCACCAAAAATCCTGAT
TTTCTCAAAGATGATTTCGAAGGTCAGCTAGAATCTGATAGATTCTTATTAATGAGTGGTGGGA
AACCATCTCGAAAGTGTGGAATCCTTTCTAGTGGAAACAACCTCTTTTTCAATGAAGATGGCTT
GCGCATGTTGATGACACGAGACCTGGATTTATCACATGCTAGATTTGTGCAGTTCTTCATGAGA
CTGGGATGTGGTAAAGGCGTTCCTGACCCCAGGAGTCAACCCGTGCTCCTACAGTATTCTCTCA
ACGGTGGCCTCTCGTGGAGTCTTCTTCAGGAGTTCCTTTTCAGCAATTCCAGCGATTACAAGGA
TGACGACGATAAGTGACTCGAG
Amino acid sequence of recombinant human R3 + R5 or R35 REELIN
protein, R3 fragment conjugated to the R5 fragment.
(SEQ ID NO: 12)
MERSGWARQTFLLALLLGATLRARAFSSTAPVLLQYSHDAGMSWFLVKEGCYPASAGKG
CEGNSRELSEPTMYHTGDFEEWTRITIVIPRSLASSKTRFRWIQESSSQKNVPPFGLDGVYISE
PCPSYCSGHGDCISGVCFCDLGYTAAQGTCVSNVPNHNEMFDRFEGKLSPLWYKITGAQVGTGC
GTLNDGKSLYFNGPGKREARTVPLDTRNIRLVQFYIQIGSKTSGITCIKPRTRNEGLIVQYSND
NGILWHLLRELDFMSFLEPQIISIDLPQDAKTPATAFRWWQPQHGKHSAQWALDDVLIGMNDSS
QTGFQDKFDGSIDDNWFFYPGGNIGLYCPYSSKGAPEEDSAMVFVSNEVGEHSITTRDLNVNEN
TIIQFEINVGCSTDSSSADPVRLEFSRDFGATWHLLLPLCYHSSSHVSSLCSTEHHPSSTYYAG
TMQGWRREVVHFGKLHLCGSVRFRWYQGFYPAGSQPVTWAIDNVYIGPQCEEMCNGQGSCINGT
KCICDPGYSGPTCKISTKNPDFLKDDFEGQLESDRFLLMSGGKPSRKCGILSSGNNLFFNEDGL
RMLMTRDLDLSHARFVQFFMRLGCGKGVPDPRSQPVLLQYSLNGGLSWSLLQEFLFSNSS

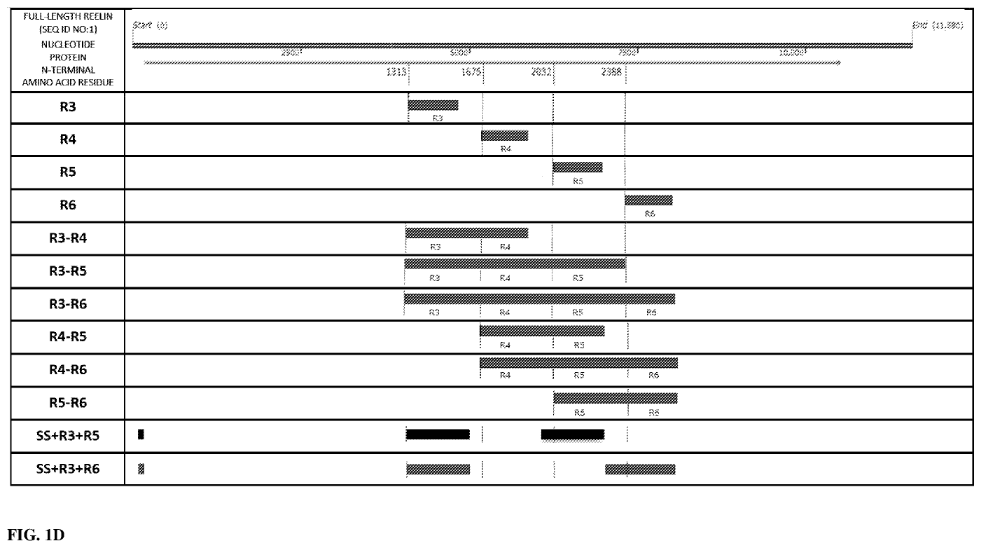

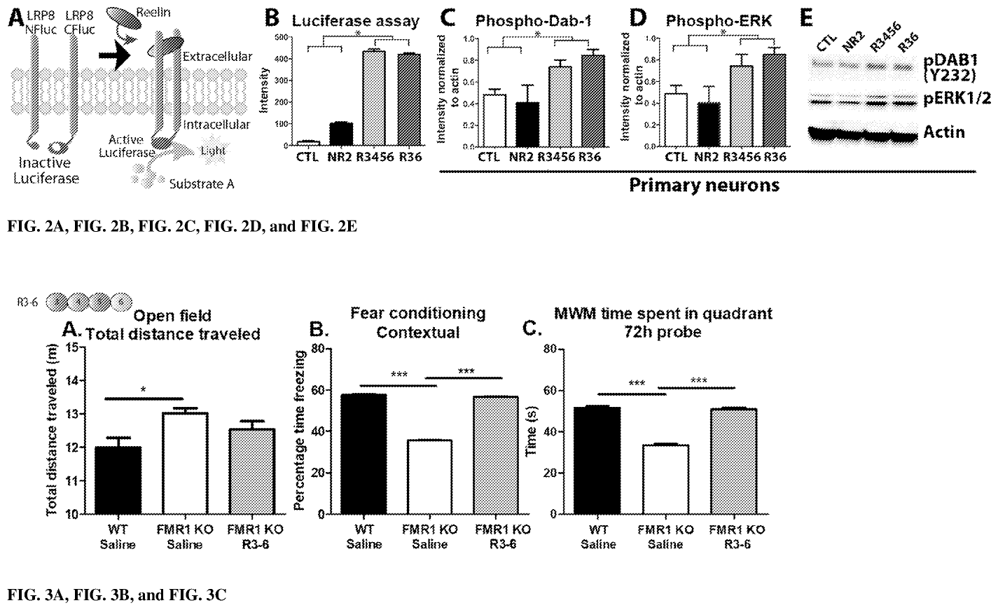

Since the R3456 fragment is too large for packaging in an AAV capsid, a the nucleotide sequence encoding a recombinant REELIN fusion protein termed R36 was generated, which contain REELIN repeat region 3 joined to REELIN repeat 6. The R36 protein fragment activates Reelin signal transduction in primary neurons, but more critically R36 can achieve the same biological effects as full-length Reelin. One innovative aspect of this disclosure is that the REELIN repeat regions 3 and 6 fusion construct (R36, also referred to herein as R3+6) can be packaged in a rAAV for injection into the CNS of subjects with Fragile X Syndrome. The R36 Reelin construct activated the Reelin receptor LRP8, as effectively as the native R3456 fragment (also referred to herein as an R3-6 fragment) (FIG. 2B). The R3+6 construct also triggered the REELIN signaling activity as indicated by both DAB1 and ERK phosphorylation in primary neuronal cultures (FIG. 2C-E). More importantly, the R36 fragment functionally rescued cognitive deficits in a mouse model of FXS and did not appear to have any adverse effects on wild type mouse behavior (FIG. 3). Identification of this smaller active REELIN protein fragment now paves the way toward novel gene therapy approaches using rAAV.

Currently there are no therapeutic treatments for FXS. Increased REELIN signaling promises to a beneficial therapeutic strategy for FXS because it should homeostatically regulate global neuronal function in the adult brain. Currently, there are no small agonist compounds for REELIN signaling, thus gene therapy offers an alternative therapeutic option that promises to provide long-term rescue of a number of the phenotypic aspects of FXS, including the disruption of glutamatergic and GABAergic pathways, LTP and dendritic spine maturation, which results in rescue of learning and memory as assessed in FMR1 KO mice.

Accordingly, in one aspect, disclosed herein are methods of treating, inhibiting, reducing, decreasing, and/or ameliorating Fragile X Syndrome, comprising administering a therapeutically effective amount of a recombinant REELIN viral vector into a patient in need thereof; where the REELIN viral vector (such as, for example, a viral vector selected from the group consisting of AAV-9, AAV-5, AAV-4, AAV-2 and AAV-1 or any variant thereof) comprises an expression construct encoding a recombinant REELIN protein fragment or native REELIN protein fragment; and where the REELIN viral vector is injected intraarterially, intravenously, intracerebrally, intraventricularly, intrathecally, subcutaneously, intradermally or intramuscularly as well as by direct injection into a tissue or organ into the patient. In certain aspects, the viral vector expresses a recombinant REELIN protein fragment selected from the group consisting of a REELIN repeat R3 protein fragment, a REELIN repeat R4 protein fragment, a REELIN repeat R5 protein fragment, a REELIN repeat R6 protein fragment, a REELIN repeat R3 through R4 protein fragment, REELIN repeat R3 through R5 protein fragment, REELIN repeat R3 through R6 protein fragment, REELIN repeat R4 through R5 protein fragment, REELIN repeat R5 through R6 protein fragment, REELIN repeat R3 joined to repeat R5 protein fragment, REELIN repeat R3 joined to repeat R6 protein fragment, REELIN repeat R4 joined to repeat R6 protein fragment, or combinations thereof; wherein R3 is repeat region 3 of full length REELIN, R4 is repeat region 4 of full length REELIN, R5 is repeat region 5 of full length REELIN, and R6 is repeat region 6 of full length REELIN. In some aspects, the REELIN viral vector can be injected into one or more specific regions of the brain, including, for example, the cortex, hippocampus, thalamus, hypothalamus, cerebellum, brain stem or spinal cord.

Also disclosed herein are methods of treating, inhibiting, reducing, decreasing, and/or ameliorating a symptom of Fragile X Syndrome (such as, for example, deficiency in dendritic spine density, diminished long-term potentiation, diminished synaptic plasticity and associative learning deficits), comprising administering a therapeutically effective amount of a recombinant REELIN viral vector, where the REELIN viral vector (such as, for example, a viral vector selected from the group consisting of AAV-9, AAV-5, AAV-4, AAV-2 and AAV-1 or any variant thereof) comprises an expression construct encoding a recombinant REELIN protein fragment or native REELIN protein fragment; wherein the REELIN viral vector is injected intraarterially, intravenously, intracerebrally, intraventricularly or intrathecally into the patient. In some aspects, the REELIN viral vector can be injected into one or more specific regions of the brain, including, for example, the cortex, hippocampus, thalamus, hypothalamus, cerebellum, brain stem or spinal cord. In certain aspects, the viral vector expresses a recombinant REELIN protein fragment selected from the group consisting of a REELIN repeat R3 protein fragment, a REELIN repeat R4 protein fragment, a REELIN repeat R5 protein fragment, a REELIN repeat R6 protein fragment, a REELIN repeat R3 through R4 protein fragment, REELIN repeat R3 through R5 protein fragment, REELIN repeat R3 through R6 protein fragment, REELIN repeat R4 through R5 protein fragment, REELIN repeat R5 through R6 protein fragment, REELIN repeat R3 joined to repeat R5 protein fragment, REELIN repeat R3 joined to repeat R6 protein fragment, REELIN repeat R4 joined to repeat R6 protein fragment, or combinations thereof; wherein R3 is repeat region 3 of full length REELIN, R4 is repeat region 4 of full length REELIN, R5 is repeat region 5 of full length REELIN, and R6 is repeat region 6 of full length REELIN.

### Delivery of the Compositions to Cells

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

### Nucleic Acid Based Delivery Systems

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, a REELIN plasmid or viral vector may contain a promoter, e.g. a CMV promoter, that drives the expression of a transgene encoding a recombinant REELIN protein fragment selected from the group consisting of, for example, R3 (Reelin repeat 3), R4 (Reelin repeat 4), R5 (Reelin repeat 5), R6 (Reelin repeat 6), R7 (Reelin repeat 7), R8 (Reelin repeat 8), R3456 (also referred to herein as R3-6 which is a fragment comprising Reelin repeat 3, Reelin repeat 4, Reelin Repeat 5 and Reelin repeat 6), R345 (also referred to herein as R3-5 which is a fragment comprising Reelin repeat 3, Reelin repeat 4, and Reelin Repeat 5), R34 (Reelin Repeat Loop Region 3-4), R35 (also referred to herein as R3+R5 or R3+5 which is a fusion or splice of R3 and R5), R36 (also referred to herein as R3+R6 or R3+6 which is a fusion or splice of R3 and R6), R456 (also referred to herein as R4-6 which is a fragment comprising Reelin repeat 4, Reelin repeat 5, and Reelin Repeat 6), R45 (Reelin Repeat Loop Region 4-5), R46 (also referred to herein as R4+R6 or R4+6 which is a fusion or splice of R4 and R6), and/or R56 (Reelin Repeat Loop Region 5-6). In certain aspects, the REELIN plasmid or viral vector is then transfected into a cell Viral vectors can be, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

### Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

### Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

### Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19 (such as, for example at AAV integration site 1 (AAVS1)). Vectors which contain this site-specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are incapable of integration into a mammalian chromosome and thus remain episomal without incurring substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors and may contain upstream elements and response elements.

The present disclosure provides a recombinant adeno-associated virus (rAAV) vector. “AAV” is an abbreviation for adeno-associated virus, and may be used to refer to the virus itself or derivatives thereof. The term covers all subtypes and both naturally occurring and recombinant forms. The abbreviation “rAAV” refers to recombinant adeno-associated virus, also referred to as a recombinant AAV vector (or “rAAV vector”) or simply, an “AAV vector.” The term “AAV” includes, for example, AAVs of various serotypes, e.g., AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10, including AAVrh10), AAVrh74, or AAV type 12 (AAV-12).

The genomic sequences of various serotypes of AAV, as well as the sequences of the native terminal repeats (TRs), Rep proteins, and capsid subunits are known in the art. Such sequences may be found in the literature or in public databases such as GenBank. See, e.g., GenBank Accession Numbers NC-002077 (AAV-1), AF063497 (AAV-1), NC-001401 (AAV-2), AF043303 (AAV-2), NC-001729 (AAV-3), NC-001829 (AAV-4), U89790 (AAV-4), NC-006152 (AAV-5), AF513851 (AAV-7), AF513852 (AAV-8), and NC-006261 (AAV-8). Methods of generating AAV viral vector suitable for ICV injection are described, for example, in the published U.S. Patent Application US2020/0384073, the content of which is incorporated by reference herein in its entirety).

“Recombinant viral vector” means a recombinant polynucleotide vector comprising one or more heterologous sequences (i.e., polynucleotide sequence not of viral origin). In the case of recombinant parvovirus vectors, the recombinant polynucleotide is flanked by at least one, preferably two, inverted terminal repeat sequences (ITRs).

In certain aspects, the REELIN repeat nucleotide sequence may have a reduced level of CpG dinucleotides that being a reduction of about 10%, 20%, 30%, 50% or more, compared with the wild type nucleic acid sequence encoding REELIN cDNA (SEQ ID NO: 1) or fragment thereof.

As used herein, the term “equivalent” refers to the effect of a AAV gene therapy on the mitigation of at least one symptom of Fragile X Syndrome as compared to a corresponding administration of the REELIN protein fragment. In certain aspects, the intracerebroventricular (ICV) injection of rAAV9-REELIN fusion protein results in the mitigation of at least one symptom of Fragile X Syndrome that is equivalent to the ICV administration of the corresponding REELIN fusion protein. In certain aspects, the intracerebroventricular (ICV) injection of rAAV9-REELIN repeat R3+R6 viral vector results in the mitigation of at least one symptom of Fragile X Syndrome that is equivalent to the ICV administration of the corresponding REELIN repeat R3+R6 protein, the R3456 REELIN repeat protein and the full length REELIN protein encoded by the nucleotide sequence of SEQ ID NO: 1. The term “equivalent” indicate the mitigation of a symptom of Fragile X Syndrome after rAAV9-REELIN R3+R6 IVC administration is within about 10, 9, 8, 7, 6, 5, 4, 3, 2, or 1% of the mitigation of the same symptom observed after the ICV administration of the corresponding R3+R6 REELIN repeat protein.

Thus, in one aspect, disclosed herein are nay of the methods of treating, inhibiting, reducing, decreasing, and/or ameliorating Fragile X Syndrome and any of the methods of treating, inhibiting, reducing, decreasing, and/or ameliorating a symptom of Fragile X Syndrome disclosed herein (such as, for example, deficiency in dendritic spine density, diminished long-term potentiation, diminished synaptic plasticity and associative learning deficits), further comprising inserting a construct expressing a transgene encoding a recombinant REELIN protein fragment or native REELIN protein fragment into a viral vector (such as, for example, a viral vector selected from the group consisting of AAV-9, AAV-5, AAV-4, and AAV-1) to form a REELIN viral vector; and injecting the Reelin vector into a subject in need thereof. In some aspects, the expression of a recombinant Reelin fragment or Reelin splice fragment is under the control of a eukaryotic promoter. Examples of suitable promoters include adenoviral promoters, such as the adenoviral major late promoter; heterologous promoters, such as the cytomegalovirus (CMV) promoter; the respiratory syncytial virus promoter; the Rous Sarcoma Virus (RSV) promoter; chicken beta-actin CBA) promoter, the CBh promoter and the CAG promoter (cytomegalovirus early enhancer element and the promoter, the first exon, and the first intron of chicken beta-actin gene and the splice acceptor of the rabbit beta-globin gene) (Alexopoulou et al., 2008, BioMed. Central Cell Biol. 9:2), and human REELIN promoter.

### Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

### Non-Viral Based Systems

The disclosed compositions can be delivered to the target cells in a variety of ways. For example, the compositions can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, for example, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a compound and a cationic liposome can be administered to the blood afferent to a target organ or inhaled into the respiratory tract to target cells of the respiratory tract. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355, the content of which is incorporated herein by reference herein in its entirety. Furthermore, the compound can be administered as a component of a microcapsule that can be targeted to specific cell types, such as macrophages, or where the diffusion of the compound or delivery of the compound from the microcapsule is designed for a specific rate or dosage.

In the methods described above which include the administration and uptake of exogenous DNA into the cells of a subject (i.e., gene transduction or transfection), delivery of the compositions to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRx Pharmaceutical Corp., Tucson, Ariz.).

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). These techniques can be used for a variety of other specific cell types. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

Nucleic acids that are delivered to cells which are to be integrated into the host cell genome, typically contain integration sequences. These sequences are often viral related sequences, particularly when viral based systems are used. These viral integration systems can also be incorporated into nucleic acids which are to be delivered using a non-nucleic acid based system of deliver, such as a liposome, so that the nucleic acid contained in the delivery system can become integrated into the host genome.

Other general techniques for integration into the host genome include, for example, systems designed to promote homologous recombination with the host genome. These systems typically rely on sequence flanking the nucleic acid to be expressed that has enough homology with a target sequence within the host cell genome that recombination between the vector nucleic acid and the target nucleic acid takes place, causing the delivered nucleic acid to be integrated into the host genome. These systems and the methods necessary to promote homologous recombination are known to those of skill in the art.

### In Vivo/Ex Vivo

As described above, the compositions can be administered in a pharmaceutically acceptable carrier and can be delivered to a subject's cells in vivo and/or ex vivo by a variety of mechanisms well known in the art (e.g., uptake of naked DNA, liposome fusion, intramuscular injection of DNA via a gene gun, endocytosis and the like).

If ex vivo methods are employed, cells or tissues can be removed and maintained outside the body according to standard protocols well known in the art. The compositions can be introduced into the cells via any gene transfer mechanism, such as, for example, calcium phosphate mediated gene delivery, electroporation, microinjection or proteoliposomes. The transduced cells can then be infused (e.g., in a pharmaceutically acceptable carrier) or homotopically transplanted back into the subject per standard methods for the cell or tissue type. Standard methods are known for transplantation or infusion of various cells into a subject.

### Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers f unction to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

### Markers

The viral vectors can include nucleic acid sequence encoding a marker product. This marker product is used to determine if the gene has been delivered to the cell and once delivered is being expressed. Preferred marker genes are the E. coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hygromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR− cells and mouse LTK− cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### Administration

As described herein, a REELIN viral vector expresses a transgene encoding a recombinant REELIN protein fragment selected from the group consisting of a REELIN repeat R3 protein fragment, a REELIN repeat R4 protein fragment, a REELIN repeat R5 protein fragment, a REELIN repeat R6 protein fragment, a REELIN repeat R3 through R4 protein fragment, REELIN repeat R3 through R5 protein fragment, REELIN repeat R3 through R6 protein fragment, REELIN repeat R4 through R5 protein fragment, REELIN repeat R5 through R6 protein fragment, REELIN repeat R3 joined to repeat R5 protein fragment, REELIN repeat R3 joined to repeat R6 protein fragment, REELIN repeat R4 joined to repeat R6 protein fragment, or combinations thereof; wherein R3 is repeat region 3 of full length REELIN, R4 is repeat region 4 of full length REELIN, R5 is repeat region 5 of full length REELIN, and R6 is repeat region 6 of full length REELIN. In certain aspect, the REELIN viral vector can be administered by parenteral injection. Alternatively, the REELIN viral vector can be injected intraarterially, intravenously, intracerebrally, or intraventricularly. In other aspects, the REELIN viral vector is bilaterally injected into the ventricles of the patient. In some aspects, the REELIN viral vector can be injected into one or more specific regions of the brain, including, for example, the cortex, hippocampus, thalamus, hypothalamus, cerebellum, brain stem or spinal cord.

In certain aspects, the REELIN viral vector can be injected at multiple location within the brain of a Fragile X Syndrome.

In certain aspects, the injection of the REELIN viral vector may be repeated as necessary to achieve a therapeutic effect.

In other aspects, the REELIN viral vector is administered in an amount sufficient to obtain a concentration of REELIN protein fragment in the CNS fluid of about 10 μM to about 5 nM. Optional concentrations include 10 μM, 15 μM, 20 μM, 25 μM, 30 μM, 3 μM 40 μM 45 μM, 50 μM, 55 μM, 60 μM, 65 μM, 70 μM, 75 μM, 80 μM, 85 μM, 90 μM, 100 μM, 110 μM, 120 μM, 130 μM, 140 μM, 150 μM, 160 μM, 170 μM, 180 μM, 190 μM, 200 μM, 220 μM, 225 μM, 240 μM, 250 μM, 270 μM, 275 μM, 280 μM, 300 μM, 320 μM, 325 μM, 340 μM, 350 μM, 370 μM, 375 μM, 380 μM, 400 μM, 420 μM, 425 μM, 440 μM, 450 μM, 470 μM, 475 μM, 480 μM, 500 μM, 520 μM, 525 μM, 540 μM, 550 μM, 570 μM, 575 μM, 580 μM, 600 μM, 620 μM, 625 μM, 640 μM, 650 μM, 670 μM, 675 μM, 680 μM, 700 μM, 720 μM, 725 μM, 740 μM, 750 μM, 770 μM, 775 μM, 780 μM, 800 μM, 820 μM, 825 μM, 840 μM, 850 μM, 870 μM, 875 μM, 880 μM, 900 μM, 920 μM, 925 μM, 940 μM, 950 μM, 970 μM, 975 μM, 980 μM, 1 nM, 1.1 nM, 1.2 nM, 1.3 nM, 1.4 nM, 1.5 nM, 1.6 nM, 1.7 nM, 1.8 nM, 1.9 nM, 2.0 nM, 2.1 nM, 2.2 nM, 2.3 nM, 2.4 nM, 2.5 nM, 2.6 nM, 2.7 nM, 2.8 nM, 2.9 nM, 3.0 nM, 3.1 nM, 3.2 nM, 3.3 nM, 3.4 nM, 3.5 nM, 3.6 nM, 3.7 nM, 3.8 nM, 3.9 nM, 4.0 nM, 4.1 nM, 4.2 nM, 4.3 nM, 4.4 nM, 4.5 nM, 4.6 nM, 4.7 nM, 4.8 nM, 4.9 nM, and 5.0 nM. For example, the therapeutic concentration can be less than 100 nM, less than 50 nM, less than 25 nM, less than 10 nM, or about 5 nM. Dosages can be calculated based on distribution of the protein in the animal body and access through the blood brain barrier.

Dosages of the REELIN viral vector expressing a REELIN repeat protein fragment will depend upon the mode of administration, the disease or condition to be treated, the individual subject's condition, the particular viral vector, and the gene to be delivered, and can be determined in a routine manner. Exemplary doses for achieving therapeutic effects are virus titers of at least about 10⁵, 10⁶, 10⁷, 10⁸, 10⁹, 10¹⁰, 10¹¹, 10¹², 10¹³, 10¹⁴, 10¹⁵ transducing units or more, preferably about 10⁸-10¹³ transducing units, yet more preferably 10¹² transducing units/kg body weight.

In one aspect, disclosed herein are any of the methods of treating, inhibiting, reducing, decreasing, and/or ameliorating Fragile X Syndrome, and any of the methods of treating, inhibiting, reducing, decreasing, and/or ameliorating a symptom of Fragile X Syndrome (such as, for example, deficiency in dendritic spine density, diminished long-term potentiation, diminished synaptic plasticity and associative learning deficits), wherein a REELIN protein fragment as disclosed herein is administered at between 1 μl and 2 μl of a 5 nM composition for each 30-36 g of patient mass.

Administration of any of the Reelin protein, fragments, splices, fusions, or vectors encoding said fragments ideally occurs a single time for life-ling treatment of the patient. However, it is understood and herein contemplated that treatment of fragile X syndrome can comprise multiple administrations. In one aspect, disclosed herein are any of the methods of treating, inhibiting, reducing, decreasing, and/or ameliorating fragile X syndrome and any of the methods of treating, inhibiting, reducing, decreasing, and/or ameliorating a symptom of fragile X syndrome, wherein the recombinant Reelin fragment or Reelin splice fragment is administered at least 1, 2, 3, 4, 5, 6, 7, 8, 9, 10, 11, 12 times per day. Half-life of the administered dose can be longer than a few hours, thus administration can be one dose every, 1, 2, 3, 4, 5, 6, 7, 8, 9, 10, 11, 12, 13, 14, 15, 16, 17, 18, 19, 20, 21, 22, 23, 24, 30, 36, 42, 48, 54, 60, 66, 72 hours, 4, 5, 6, 7, 8, 9, 10, 11, 12, 13, 14, 15, 16, 17, 18, 19, 20, 21, 22, 23, 24, 25, 26, 27, 28, 29, 30, 31, 35, 40, 45, 50, 55, 58, 59, 60, 61, 62 days, 3, 4, 5, 6, 7, 8, 9, 10, 11, 12, 18, 24 months, 3, 4, 5, 6, 7, 8, 9, 10, 11, 12, 13, 14, 15, 16, 17, 18, 19, 20, 21, 22, 23, 24, 25 or more years for the life of the patient.

### Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition is generally characterized by injection. Injectables can be prepared in conventional forms, either as liquid solutions or suspensions, solid forms suitable for solution of suspension in liquid prior to injection, or as emulsions.

The materials may be in solution, suspension (for example, incorporated into microparticles or liposomes). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes, receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### Pharmaceutically Acceptable Carriers

The REELN and REELN fragment comprising compositions disclosed herein, (including but not limited to viral vectors) compositions, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

### Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products.

### Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular Reelin, Reelin fragments, Reelin splice, or Reelin fusion is disclosed and discussed and a number of modifications that can be made to a number of molecules including the Reelin, Reelin fragments, Reelin splice, or Reelin fusion are discussed, specifically contemplated is each and every combination and permutation of Reelin, Reelin fragments, Reelin splice, or Reelin fusion and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Also disclosed herein are compositions for using in any of the methods of treating, inhibiting, reducing, decreasing, and/or ameliorating Fragile X Syndrome a disease or disorder of the nervous system of any preceding aspect and methods of treating, inhibiting, reducing, decreasing, and/or ameliorating a symptom of a Fragile X Syndrome disease or disorder of the nervous system of any preceding aspect.

In one aspect, disclosed herein are compositions comprising a recombinant REELIN protein fragment or a native REELIN protein fragment; wherein the recombinant REELIN protein fragment is selected from the group consisting of a REELIN repeat R3 protein fragment, a REELIN repeat R4 protein fragment, a REELIN repeat R5 protein fragment, a REELIN repeat R6 protein fragment, a REELIN repeat R3 through R4 protein fragment, a REELIN repeat R3 through R5 protein fragment, a REELIN repeat R3 through R6 protein fragment, a REELIN repeat R4 through R5 protein fragment, a REELIN repeat R5 through R6 protein fragment, a REELIN repeat R3 joined to repeat R5 protein fragment, a REELIN repeat R3 joined to repeat R6 protein fragment, a REELIN repeat R4 joined to repeat R6 protein fragment, or combinations thereof; wherein R3 is repeat region 3 of full length REELIN, R4 is repeat region 4 of full length REELIN, R5 is repeat region 5 of full length REELIN, and R6 is repeat region 6 of full length REELIN.

In another aspect, disclosed herein are compositions comprising a REELIN viral vector viral having a transgene for expressing a recombinant REELIN protein fragment or a native REELIN protein fragment, wherein the recombinant REELIN protein fragment is selected from the group consisting of a REELIN repeat R3 protein fragment, a REELIN repeat R4 protein fragment, a REELIN repeat R5 protein fragment, a REELIN repeat R6 protein fragment, a REELIN repeat R3 through R4 protein fragment, a REELIN repeat R3 through R5 protein fragment, a REELIN repeat R3 through R6 protein fragment, a REELIN repeat R4 through R5 protein fragment, a REELIN repeat R5 through R6 protein fragment, a REELIN repeat R3 joined to repeat R5 protein fragment, a REELIN repeat R3 joined to repeat R6 protein fragment, a REELIN repeat R4 joined to repeat R6 protein fragment, or combinations thereof; wherein R3 is repeat region 3 of full length REELIN, R4 is repeat region 4 of full length REELIN, R5 is repeat region 5 of full length REELIN, and R6 is repeat region 6 of full length REELIN.

In some aspects, the REELIN viral vector can be injected into one or more specific regions of the brain, including, for example, the cortex, hippocampus, thalamus, hypothalamus, cerebellum, brain stem or spinal cord. In some aspects, the expression of the recombinant REELIN fragment can be under the control of a eukaryotic promotor, e.g., a CMV promoter.

### Homology/Identity

It is understood that one way to define any known variants and derivatives or those that might arise, of the disclosed genes and proteins herein is through defining the variants and derivatives in terms of homology to specific known sequences. For example, SEQ ID NO: 9 sets forth a particular sequence of a Reelin repeat R3+R6 fragment (i.e., a fusion of R3+R6 also referred to herein as R3+6 or R36) and SEQ ID NO: 10 sets forth a particular sequence of the protein encoded by SEQ ID NO: 9, an R3+6 peptide. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

### Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example, if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### Nucleic Acids

There are a variety of molecules disclosed herein that are nucleic acid based, including for example the nucleic acids that encode, for example Reelin or fragments thereof, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U.

### Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

### Peptides

### Protein Variants

As discussed herein there are numerous variants of the Reelin protein and Reelin fragments, splices, and fusion that are known and herein contemplated. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intersequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues but can occur at several different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2
Amino Acid Substitutions
Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Identification of REELIN Protein Fragments Having Signaling Activity

The Western blot depicted in FIG. 1C demonstrates that the amount of REELIN protein is reduced in FMR1−/− mice at compared to wild type mice. Hence, the loss in REELIN activity may be a contributing factor in FXS and REELIN gene therapy approaches to restore REELIN activity may be able to treat the neurological deficits associated with FXS disease.

The use of a protein therapy (repeated injections of REELIN protein or its fragments into patients) would be cost restrictive and would involve repeated lumbar injections. Although the length of time a single Reelin injection remains effective is unknown, it is certainly not long-term. A sustained, long-term administration could only be achieved via gene therapy. The gene therapy vector adeno-associated virus (AAV) has become a preferred vector for the CNS and shows great promise and safety in the treatment of a number of neurological diseases. A major caveat of the technology is the constraint on the size of the transgene that can be expressed.

To identify REELIN fragments that are more amenable to packaging for AAV gene therapy, a number of truncated Reelin fragments were initially screened in an in vitro cell binding assay (FIG. 2A) that tests for dimerization of the Reelin receptors in the presence of the different REELIN protein fragments. The assay consists of cells expressing two membrane bound recombinant receptor and luciferase fusion proteins; LRP8 fused with a N-terminal fragment of luciferase and an LRP8 fused to the C-terminus of luciferase (FIG. 2A). Luciferase activity is only observed when the two LRP8 fusion proteins dimerize. Thus, the presence of ligand-receptor interaction is indicated by increased luminescence.

Although activity from a number of REELIN fragments was observed, a recombinant REELIN fragment consisting of repeat R3 joined to repeat R6 (abbreviated as R36 or R3+R6 REELIN repeat protein fragment having the amino acid sequence of SEQ ID NO: 10 MW: 66 kD, was as efficacious as the in vivo R3456 REELIN protein fragment in both the in vitro binding assay and in the activation of Reelin signaling in primary neurons (DAB1 and ERK phosphorylation) (FIG. 2B-E). This supports the notion that not only the R3-6 fragment is an active signaling molecule in vivo, but that the R3+6 REELIN protein fragment can function like the R3-6 fragment.

### Example 2: ICV Injection of the R3+R6 REELIN Protein Fragments into a Mouse Model of Fragile X Syndrome

In order to determine in vivo efficacy, R36 functionality was tested in a mouse model of Fragile X syndrome (e.g., FMR1^{tm1Cgr} knock out mice available from Jackson Labs, ME). Fragile X syndrome is a developmental disease which exhibit dysregulation of synaptic function and cognitive impairments. FXS mice lack a functional FMR1 gene, which leads to a deficiency in FMRP (Fragile X Mental Retardation Protein) expression leading to learning deficits and decreased synaptic function. Data shows a single intracerebroventricular (ICV) injection of the R3456 REELIN protein fragment (R3-R6; 2 μL at 5 nM) into 12-14-week-old FXS male mice (n=7/group), rescued neurological deficits in FXS mice as compared to saline injected mice (FIG. 3) including deficits observed in contextual fear-conditioning (FIG. 3B) and deficits in 24 h and 72 h probe trials of Morris water maze (FIG. 3C).

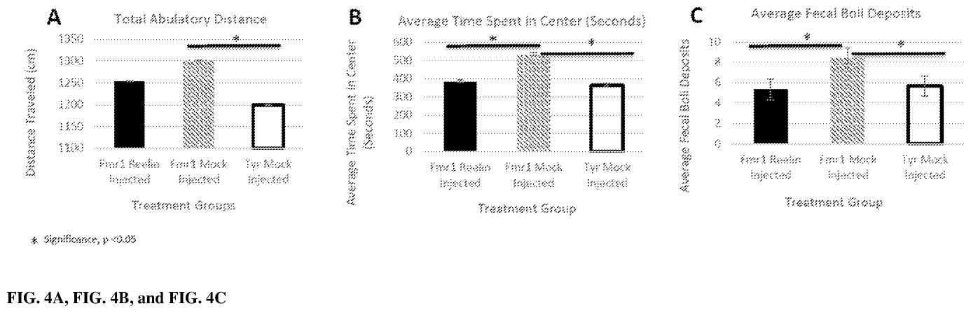

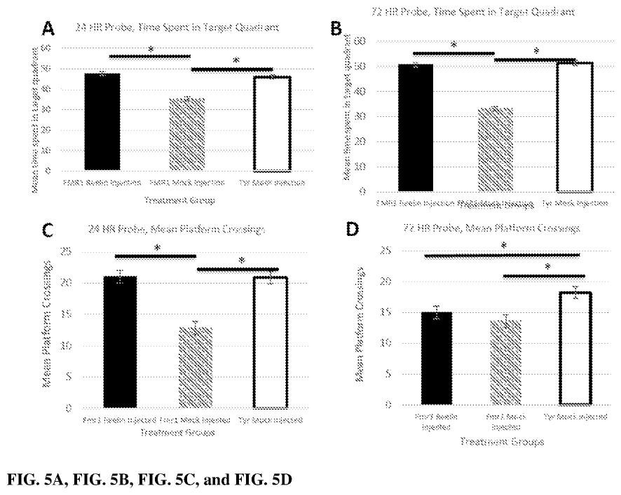

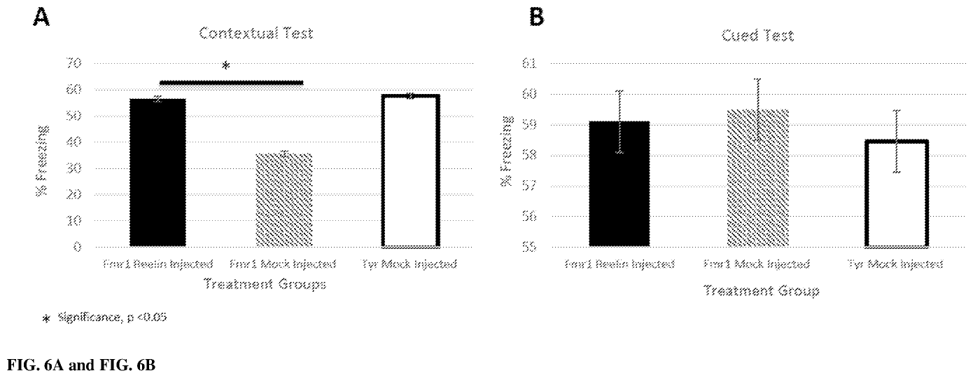

ICV administration of the R3456 REELIN protein fragment also rescued locomotor activity in FMR1 KO mice (FIG. 4) as well as spatial learning using a hidden platform water maze (FIG. 5) and associative learning deficit using the same hidden platform water maze (FIG. 6).

### Example 3: Comparison of the Activity of rAAV-9-R36 and R36 REELIN Protein Fragments in a Mouse Model of Fragile X Syndrome

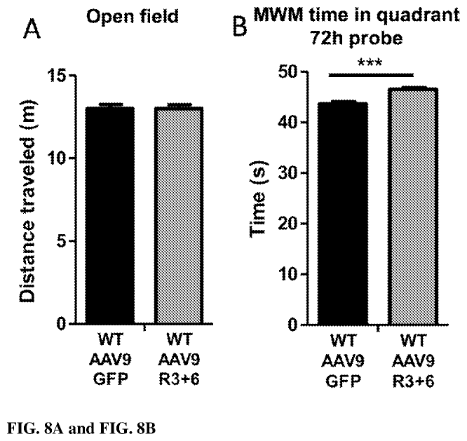

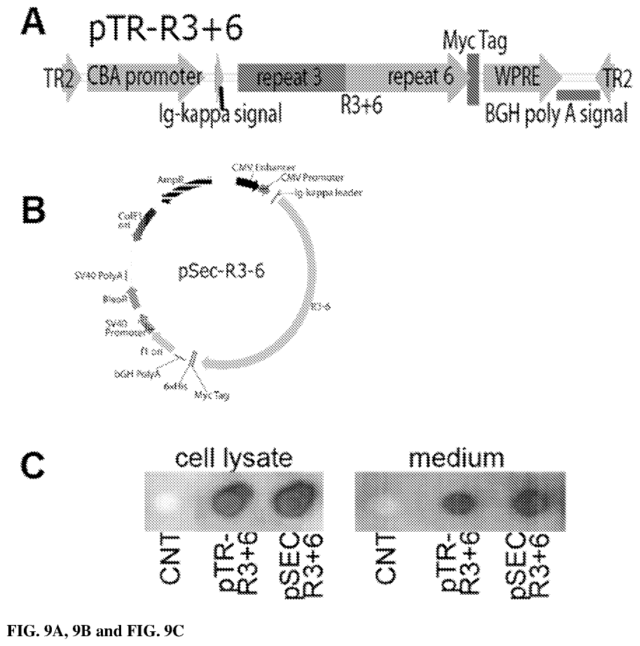

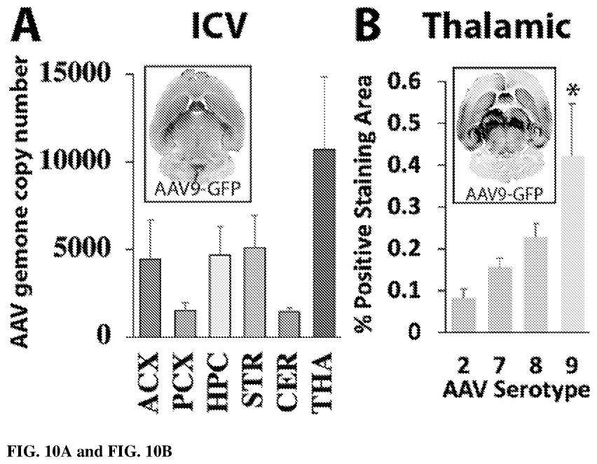

WT and FXS male mice (n=7-8/group) were injected ICV with either saline, R36 REELIN protein fragment (2 μL at 5 nM), or with rAAV9-R36 viral vector expressing the R36 REELIN protein fragment (see FIG. 9A). Saline and R36 protein injected mice were tested two days after surgery and compared directly with the R3456 injected animals above (FIG. 3). rAAV9-R36 injected animals were tested after 6 weeks to allow for stable transgene expression, along with a cohort of control mice. No adverse events were observed in WT mice due to expression of the REELIN viral vector (see FIG. 8A), including sickness type behaviors (e.g hunching, loss of weight) or changes in activity in the open field task following ˜2 months of transgene expression.

A single ICV administration of R3-6 or R3+6 REELIN repeat protein fragments or rAAV9-R36 were able to rescue both learning and memory deficits in FMR1−/− mice (FIGS. 3,5 and 7).

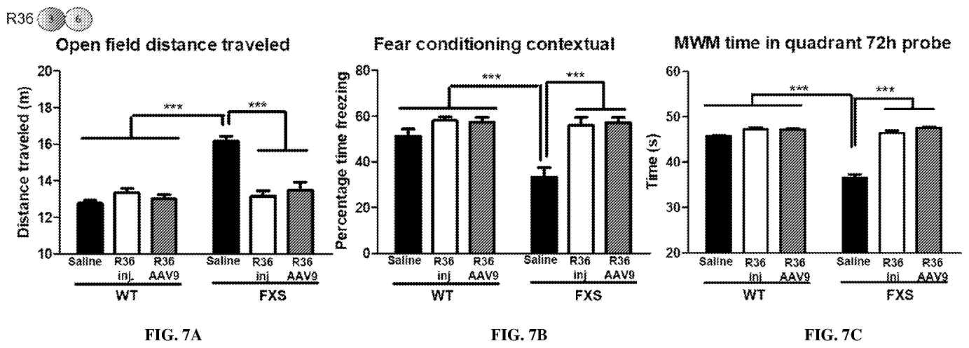

Hyperactivity was monitored by measuring an animals' activity levels in open field VersaMax chambers (Accuscan Instruments, Columbus, Ohio). Locomotor activity was detected by photobeam breaks as the animal crossed each beam. The animal was placed in the center of the field and left undisturbed for a period of time (20 min to 2 hr) to measure its spontaneous activity in a novel environment. Measurements used to assess locomotor activity included: horizontal activity, total distance traveled, vertical activity (rearing events—animal raises up on hindlimbs), rotation, stereotypy, and distance traveled in the center compared to total distance traveled (center:total distance ratio). Hyperactivity of FMR1 KO mice (increased distance traveled) in the open field, was partially rescued with R3-6 (FIG. 3A) and normalized to control with R3+6 injection (FIG. 7A).

Both R3-6 and R3+6 administration rescued FMR1 KO freezing in the contextual fear-conditioning task (FIG. 3B, FIG. 7B). No deficits were seen in the cued response for the FMR1 KO mice as seen by others. Although no significant differences were observed during training in the Morris water maze, FMR1 KO mice showed significant impairment in recall 24 h and 72 h post training (FIG. 3C, FIG. 7C). Injection of R3-6 REELIN repeat protein fragment or R3+6 REELIN repeat protein fragment or rAAV9-R36 into FMR1 KO mice was able to restore the time spent in target quadrant to wild type levels at both 24 h and 72 h post training, demonstrating cognitive improvement (FIG. 3C, FIG. 7C).

These data indicate that both R3-6 and R3+6 REELIN repeat protein fragments and the rAAV9-R36 viral vector are sufficient to induce Reelin signaling and restore deficits in the FMR1 KO mice, offering a viable therapy to treat FXS.

These data indicate three important pieces of information, (1) R36 is a fully active substitute for full length Reelin in vivo, (2) R36 showed robust behavioral phenotype rescue in a test model of synaptic dysregulation (FXS mice), and (3) R36 can be expressed from a rAAV and elicit the same activity and therapeutic effects as R3456 in vivo. Thus, rAAV9-R36 construct is a viable approach to increase Reelin signaling within the CNS.

In a preliminary evaluation of the rAAV9-R3+6 viral vector, wild type mice were injected with either rAAV9-R3+6 or rAAV9-GFP. Importantly, no adverse events were observed as a result of the injections such as sickness type behaviors (e.g hunching, loss of weight) or locomotor activity in open field during the 2 months of expression (FIG. 8A). In fact, in learning and memory tasks a small but significant improvement in cognition was observed as shown by increased time spent in quadrant during MWM 72 h probe trial (FIG. 8B), which is consistent with previous findings on REELIN protein injections into wild type mice (U.S. Patent Application No. 2019/0169246, the content of which is incorporated by reference herein in its entirety).

### Example 4: Rescue of Synaptic Proteins and Spine Morphology with rAAV9-R3+6

It is well established that FMR1 KO mice show dysregulation of proteins involved in glutamatergic and GABAergic neurotransmission as well as decreased dendritic spine maturation. Considering the known effects of Reelin on neuronal plasticity, administration of rAAV9-R3+6 was tested if it could restore critical synaptic proteins that are altered in FXS, such as Arc/Arg3.1, CamKII, PSD95, GABAergic pathway (GAD67, GAT, GABA receptor subunits), glutamatergic pathway (AMPA and NMDA receptors) as well as improve spine maturation.

A transgene encoding the R3+6 REELIN repeat protein fragment was cloned downstream of a CMV chicken beta-actin hybrid promoter (termed CBA or CAG) in the AAV vector pTR 12.1-MCSW (FIG. 9A). Expression and secretion from the AAV vector was confirmed in HEK-293 cells as indicated by positive anti-myc tag detection in the cell lysate and media of transfected cells (FIG. 9B).

### Example 5: Behavioral Rescue of FMR1 KO Mice with rAAV9-R3+6

A role for REELIN protein fragments as a potential therapeutic for the treatment of Fragile X Syndrome is supported by 1) the observed reduction in REELIN protein levels in FMR1 KO mice (FIG. 1) and 2) rescue of behavioral deficits in FMR1 KO mice with single injection of R3-6 and R3+R6 REELIN protein fragments or rAAV9-R3+R6 viral vector (FIGS. 7 & 8). AAV delivery of REELIN fragment R3+6 in the brain of FMR1 KO mice provides long-term benefits to learning and memory.

An equal number of male and female FMR1 KO mice (n=20/group) and WT mice can be injected with rAAV9-R3+6 or GFP ICV at 3 months of age. Behavioral testing can be performed two months after injection, to allow for sustained expression of Reelin R3+6 fragment and functional recovery. Behavioral Analysis: Equal numbers of both male and female mice can be examined to account for any potential gender differences. Previous studies typically use male FMR1 KO mice because of the increased prevalence and severity of FXS in male patients, although it has been shown that both male and female mice equally show behavioral deficits. All groups of mice can be tested in open field, Morris water maze and fear conditioning. However, behavioral testing can be expanded to include other tasks such as novel object recognition, Y-maze, rotarod, social mouse interaction, forced swim and anxiety in elevated plus maze. These tasks are important, even if no phenotypic deficit is expected in the FMR1 KO mice compared to WT, because it was important to determine if increased Reelin signaling with rAAV9-R3+6, over the ˜3 months expression, has any negative effects on behavior (a first approximation of safety). Testing can be performed over a 1-month period to allow for rest periods between tasks, and with the tasks being performed from least stressful (open field) to most stressful (fear conditioning). All researchers involved in testing can be blinded to genotype and treatment group, with animals identified only by a unique identification number assigned at weaning by ear notches.

### Statistics:

Analysis of measures can be performed using one-way (comparison of R3+6 treatment effect to GFP treated) or two-way ANOVA (treatment and sex differences). Repeated measure can be performed on tasks like rotarod and water maze which require multiple training/trials. Post-hoc means comparisons for significant ANOVA measures can use Tukey's HSD using GraphPad Prism.

### Example 6: Rescue of Electrophysiological Deficits in FMR1 KO Mice With rAAV9-R3+6

The rAAV9-R3+R6 viral vector can rescue FMR1 KO mice electrophysiologic deficits. FMR1 KO mice have been shown to have electrophysiologic deficits, with decreased LTP observed in the anterior cingulate cortex as well as the dentate gyrus of the hippocampus. Interestingly, there seems to be no reduction in LTP in the CA1 region of the hippocampus in young mice (2-12 months of age), but there is a loss of theta-burst induced LTP in aged mice (>12 mo). LTD in the CA1 was also enhanced in FMR1 KO mice. These disruptions are thought to contribute to the phenotype of FXS. The mechanism of Reelin in learning and memory can be through stabilization of LTP and dendritic spines. The data demonstrated increased learning and memory in FMR1 KO after the administration of R3-6 and R3+R6 REELIN protein fragments or rAAV9-R3+R6 viral vector, which can be attributed to a restoration of LTP and LTD in these animals. REELIN protein administration has been shown to rescue LTP and dendritic spine deficits.

In this phase of experiments mice can be divided into 2 subgroups for LTP (n=10) and LTD (n=10). WT mice injected with rAAV9-GFP can be used as controls for all measures. All experimenters can be blinded to the genotype and treatment condition of the mice.

### Electrophysiology:

Hippocampal slice preparation and electrophysiology can be performed. Input/output responses can be used to assess overall baseline synaptic transmission. LTP can be induced using a standard 2 trains of 100 Hz frequency stimulation for 1 sec with each train separated by a 20 s interval. Experimental results can be obtained from those slices that exhibited stable baseline synaptic transmission for a minimum of 30 minutes prior to the delivery of the LTP-inducing stimulus. LTD can be examined by induction by bathing hippocampal slices in 100 μM (S)3,5-DHPG for 10 min, after establishment of a stable baseline. LTP and LTD recordings can be obtained from area CA1 stratum radiatum with stimulation from the CA3 Schaffer collaterals. Electrophysiology can be analyzed using a repeated measures and one-way ANOVA followed by Tukey's Multiple Comparison post-hoc test. Significance set to p<0.05.

### REFERENCES

1. Niu M, Han Y, Dy A B C, Du J, Jin H, Qin J, Zhang J, Li Q, Hagerman R J. Autism Symptoms in Fragile X Syndrome. J Child Neurol. 2017; 32(10):903-9. Epub 2017 Jun. 16. doi: 10.1177/0883073817712875. PubMed PMID: 28617074.
2. Saldarriaga W, Tassone F, Gonzalez-Teshima L Y, Forero-Forero J V, Ayala-Zapata S, Hagerman R. Fragile X syndrome. Colomb Med (Cali). 2014; 45(4):190-8. Epub 2015 Mar. 15. PubMed PMID: 25767309; PMCID: PMC4350386.
3. Ding Q, Sethna F, Wang H. Behavioral analysis of male and female Fmr1 knockout mice on C57BL/6 background. Behavioural brain research. 2014; 271:72-8. Epub 2014 Jun. 3. doi: 10.1016/j.bbr.2014.05.046. PubMed PMID: 24886775; PMCID: PMC4104211.
4. Chen L, Yun S W, Seto J, Liu W, Toth M. The fragile X mental retardation protein binds and regulates a novel class of mRNAs containing U rich target sequences. Neuroscience. 2003; 120(4):1005-17. Epub 2003 Aug. 21. doi: 10.1016/s0306-4522(03)00406-8. PubMed PMID: 12927206.
5. Brown V, Jin P, Ceman S, Darnell J C, O'Donnell W T, Tenenbaum S A, Jin X, Feng Y, Wilkinson K D, Keene J D, Darnell R B, Warren S T. Microarray identification of FMRP-associated brain mRNAs and altered mRNA translational profiles in fragile X syndrome. Cell. 2001; 107(4):477-87. Epub 2001 Nov. 24. doi: 10.1016/s0092-8674(01)00568-2. PubMed PMID: 11719188.
6. Miyashiro K Y, Beckel-Mitchener A, Purk T P, Becker K G, Barret T, Liu L, Carbonetto S, Weiler I J, Greenough W T, Eberwine J. RNA cargoes associating with FMRP reveal deficits in cellular functioning in Fmr1 null mice. Neuron. 2003; 37(3):417-31. Epub 2003 Feb. 11. PubMed PMID: 12575950.
7. Paluszkiewicz S M, Martin B S, Huntsman M M. Fragile X syndrome: the GABAergic system and circuit dysfunction. Dev Neurosci. 2011; 33(5):349-64. Epub 2011 Sep. 22. doi: 10.1159/000329420. PubMed PMID: 21934270; PMCID: PMC3254035.
8. Kazdoba T M, Leach P T, Silverman J L, Crawley J N. Modeling fragile X syndrome in the Fmr1 knockout mouse. Intractable Rare Dis Res. 2014; 3(4):118-33. Epub 2015 Jan. 22. doi: 10.5582/irdr.2014.01024. PubMed PMID: 25606362; PMCID: PMC4298642.
9. Fatemi S H, Earle J A, McMenomy T. Reduction in Reelin immunoreactivity in hippocampus of subjects with schizophrenia, bipolar disorder and major depression. Mol Psychiatry. 2000; 5(6):654-63, 571. PubMed PMID: 11126396.
10. Fatemi S H, Snow A V, Stary J M, Araghi-Niknam M, Reutiman T J, Lee S, Brooks A I, Pearce D A. Reelin signaling is impaired in autism. Biol Psychiatry. 2005; 57(7):777-87. doi: 10.1016/j.biopsych.2004.12.018. PubMed PMID: 15820235.
11. Mohammadi A, Rashidi E, Amooeian V G. Brain, blood, cerebrospinal fluid, and serum biomarkers in schizophrenia. Psychiatry Res. 2018; 265:25-38. Epub 2018 Apr. 24. doi: 10.1016/j.psychres.2018.04.036. PubMed PMID: 29680514.
12. Folsom T D, Fatemi S H. The involvement of Reelin in neurodevelopmental disorders. Neuropharmacology. 2013; 68:122-35. Epub 2012 Sep. 18. doi: 10.1016/j.neuropharm.2012.08.015. PubMed PMID: 22981949; PMCID: PMC3632377.
13. Ishii K, Kubo K I, Nakajima K. Reelin and Neuropsychiatric Disorders. Frontiers in cellular neuroscience. 2016; 10:229. Epub 2016 Nov. 3. doi: 10.3389/fncel.2016.00229. PubMed PMID: 27803648; PMCID: PMC5067484.
14. Bosch C, Masachs N, Exposito-Alonso D, Martinez A, Teixeira C M, Fernaud I, Pujadas L, Ulloa F, Comella J X, DeFelipe J, Merchan-Perez A, Soriano E. Reelin Regulates the Maturation of Dendritic Spines, Synaptogenesis and Glial Ensheathment of Newborn Granule Cells. Cereb Cortex. 2016; 26(11):4282-98. Epub 2016 Sep. 15. doi: 10.1093/cercor/bhw216. PubMed PMID: 27624722; PMCID: PMC5066826.
15. Bosch C, Muhaisen A, Pujadas L, Soriano E, Martinez A. Reelin Exerts Structural, Biochemical and Transcriptional Regulation Over Presynaptic and Postsynaptic Elements in the Adult Hippocampus. Frontiers in cellular neuroscience. 2016; 10:138. Epub 2016 Jun. 16. doi: 10.3389/fncel.2016.00138. PubMed PMID: 27303269; PMCID: PMC4884741.
16. Qiu S, Weeber E J. Reelin signaling facilitates maturation of CA1 glutamatergic synapses. J Neurophysiol. 2007; 97(3):2312-21. Epub 2007 Jan. 19. doi: 10.1152/jn.00869.2006. PubMed PMID: 17229826.
17. Senkov O, Andjus P, Radenovic L, Soriano E, Dityatev A. Neural ECM molecules in synaptic plasticity, learning, and memory. Prog Brain Res. 2014; 214:53-80. Epub 2014 Nov. 21. doi: 10.1016/B978-0-444-63486-3.00003-7. PubMed PMID: 25410353.
18. Levy A D, Omar M H, Koleske A J. Extracellular matrix control of dendritic spine and synapse structure and plasticity in adulthood. Front Neuroanat. 2014; 8:116. Epub 2014 Nov. 5. doi: 10.3389/fnana.2014.00116. PubMed PMID: 25368556; PMCID: PMC4202714.
19. Niu S, Renfro A, Quattrocchi C C, Sheldon M, D'Arcangelo G. Reelin promotes hippocampal dendrite development through the VLDLR/ApoER2-Dab1 pathway. Neuron. 2004; 41(1):71-84. Epub 2004 Jan. 13. PubMed PMID: 14715136.
20. Rogers J T, Rusiana I, Trotter J, Zhao L, Donaldson E, Pak D T, Babus L W, Peters M, Banko J L, Chavis P, Rebeck G W, Hoe H S, Weeber E J. Reelin supplementation enhances cognitive ability, synaptic plasticity, and dendritic spine density. Learn Mem. 2011; 18(9):558-64. Epub 2011 Aug. 20. doi: 10.1101/lm.2153511. PubMed PMID: 21852430; PMCID: PMC3166788.
21. Rogers J T, Zhao L, Trotter J H, Rusiana I, Peters M M, Li Q, Donaldson E, Banko J L, Keenoy K E, Rebeck G W, Hoe H S, D'Arcangelo G, Weeber E J. Reelin supplementation recovers sensorimotor gating, synaptic plasticity and associative learning deficits in the heterozygous reeler mouse. J Psychopharmacol. 2013; 27(4):386-95. doi: 10.1177/0269881112463468. PubMed PMID: 23104248; PMCID: PMC3820099.
22. Royaux I, Lambert de Rouvroit C, D'Arcangelo G, Demirov D, Goffinet A M. Genomic organization of the mouse reelin gene. Genomics. 1997; 46(2):240-50. Epub 1998 Jan. 7. PubMed PMID: 9417911.
23. Jossin Y, Ignatova N, Hiesberger T, Herz J, Lambert de Rouvroit C, Goffinet A M. The central fragment of Reelin, generated by proteolytic processing in vivo, is critical to its function during cortical plate development. J Neurosci. 2004; 24(2):514-21. doi: 10.1523/JNEUROSCI.3408-03.2004. PubMed PMID: 14724251.
24. Turner G, Webb T, Wake S, Robinson H. Prevalence of fragile X syndrome. Am J Med Genet. 1996; 64(1):196-7. Epub 1996 Jul. 12. doi: 10.1002/(SICI)1096-8628(19960712)64:1<196::AID-AJMG35>3.0.CO;2-G. PubMed PMID: 8826475.
25. O'Donnell W T, Warren S T. A decade of molecular studies of fragile X syndrome. Annu Rev Neurosci. 2002; 25:315-38. Epub 2002 Jun. 8. doi: 10.1146/annurev.neuro.25.112701.142909. PubMed PMID: 12052912.
26. Shang Y, Wang H, Mercaldo V, Li X, Chen T, Zhuo M. Fragile X mental retardation protein is required for chemically-induced long-term potentiation of the hippocampus in adult mice. J Neurochem. 2009; 111(3):635-46. Epub 2009 Aug. 8. doi: 10.1111/j.1471-4159.2009.06314.x. PubMed PMID: 19659572.
27. Thelen M P, Kye M J. The Role of RNA Binding Proteins for Local mRNA Translation: Implications in Neurological Disorders. Front Mol Biosci. 2019; 6:161. Epub 2020 Feb. 6. doi: 10.3389/fmolb.2019.00161. PubMed PMID: 32010708; PMCID: PMC6974540.
28. Zafarullah M, Tassone F. Molecular Biomarkers in Fragile X Syndrome. Brain Sci. 2019; 9(5). Epub 2019 May 1. doi: 10.3390/brainsci9050096. PubMed PMID: 31035599; PMCID: PMC6562871.
29. Sung Y J, Weiler I J, Greenough W T, Denman R B. Selectively enriched mRNAs in rat synaptoneurosomes. Brain Res Mol Brain Res. 2004; 126(1):81-7. Epub 2004 Jun. 23. doi: 10.1016/j.molbrainres.2004.03.013. PubMed PMID: 15207920.
30. Nakamoto M, Nalavadi V, Epstein M P, Narayanan U, Bassell G J, Warren S T. Fragile X mental retardation protein deficiency leads to excessive mGluR5-dependent internalization of AMPA receptors. Proc Natl Acad Sci USA. 2007; 104(39):15537-42. Epub 2007 Sep. 21. doi: 10.1073/pnas.0707484104. PubMed PMID: 17881561; PMCID: PMC2000537.
31. Huber K M, Gallagher S M, Warren S T, Bear M F. Altered synaptic plasticity in a mouse model of fragile X mental retardation. Proc Natl Acad Sci USA. 2002; 99(11):7746-50. Epub 2002 May 29. doi: 10.1073/pnas.122205699. PubMed PMID: 12032354; PMCID: PMC124340.
32. Auerbach B D, Bear M F. Loss of the fragile X mental retardation protein decouples metabotropic glutamate receptor dependent priming of long-term potentiation from protein synthesis. J Neurophysiol. 2010; 104(2):1047-51. Epub 2010 Jun. 18. doi: 10.1152/jn.00449.2010. PubMed PMID: 20554840; PMCID: PMC2934918.
33. Sidorov M S, Auerbach B D, Bear M F. Fragile X mental retardation protein and synaptic plasticity. Mol Brain. 2013; 6:15. Epub 2013 Apr. 10. doi: 10.1186/1756-6606-6-15. PubMed PMID: 23566911; PMCID: PMC3636002.
34. Michalon A, Sidorov M, Ballard T M, Ozmen L, Spooren W, Wettstein J G, Jaeschke G, Bear M F, Lindemann L. Chronic pharmacological mGlu5 inhibition corrects fragile X in adult mice. Neuron. 2012; 74(1):49-56. Epub 2012 Apr. 17. doi: 10.1016/j.neuron.2012.03.009. PubMed PMID: 22500629.
35. Michalon A, Bruns A, Risterucci C, Honer M, Ballard T M, Ozmen L, Jaeschke G, Wettstein J G, von Kienlin M, Kunnecke B, Lindemann L. Chronic metabotropic glutamate receptor 5 inhibition corrects local alterations of brain activity and improves cognitive performance in fragile X mice. Biol Psychiatry. 2014; 75(3):189-97. Epub 2013 Aug. 6. doi: 10.1016/j.biopsych.2013.05.038. PubMed PMID: 23910948.
36. Bostrom C A, Majaess N M, Morch K, White E, Eadie B D, Christie B R. Rescue of NMDAR-dependent synaptic plasticity in Fmr1 knock-out mice. Cereb Cortex. 2015; 25(1):271-9. Epub 2013 Aug. 24. doi: 10.1093/cercor/bht237. PubMed PMID: 23968838.
37. Krueger D D, Osterweil E K, Chen S P, Tye L D, Bear M F. Cognitive dysfunction and prefrontal synaptic abnormalities in a mouse model of fragile X syndrome. Proc Natl Acad Sci USA. 2011; 108(6):2587-92. Epub 2011 Jan. 26. doi: 10.1073/pnas.1013855108. PubMed PMID: 21262808; PMCID: PMC3038768.
38. Dictenberg J B, Swanger S A, Antar L N, Singer R H, Bassell G J. A direct role for FMRP in activity-dependent dendritic mRNA transport links filopodial-spine morphogenesis to fragile X syndrome. Dev Cell. 2008; 14(6):926-39. Epub 2008 Jun. 10. doi: 10.1016/j.devce1.2008.04.003. PubMed PMID: 18539120; PMCID: PMC2453222.
39. Weiler I J, Greenough W T. Synaptic synthesis of the Fragile X protein: possible involvement in synapse maturation and elimination. Am J Med Genet. 1999; 83(4):248-52. Epub 1999 Apr. 20. doi: 10.1002/(sici)1096-8628(19990402)83:4<248::aid-ajmg3>3.0.co;2-1. PubMed PMID: 10208156.
40. Harlow E G, Till S M, Russell T A, Wijetunge L S, Kind P, Contractor A. Critical period plasticity is disrupted in the barrel cortex of FMR1 knockout mice. Neuron. 2010; 65(3):385-98. Epub 2010 Feb. 18. doi: 10.1016/j.neuron.2010.01.024. PubMed PMID: 20159451; PMCID: PMC2825250.
41. Cuchillo-Ibanez I, Balmaceda V, Mata-Balaguer T, Lopez-Font I, Saez-Valero J. Reelin in Alzheimer's Disease, Increased Levels but Impaired Signaling: When More is Less. J Alzheimers Dis. 2016; 52(2):403-16. Epub 2016 Apr. 1. doi: 10.3233/JAD-151193. PubMed PMID: 27031488.
42. Trommsdorff M, Gotthardt M, Hiesberger T, Shelton J, Stockinger W, Nimpf J, Hammer R E, Richardson J A, Herz J. Reeler/Disabled-like disruption of neuronal migration in knockout mice lacking the VLDL receptor and ApoE receptor 2. Cell. 1999; 97(6):689-701. PubMed PMID: 10380922.
43. Weeber E J, Beffert U, Jones C, Christian J M, Forster E, Sweatt J D, Herz J. Reelin and ApoE receptors cooperate to enhance hippocampal synaptic plasticity and learning. J Biol Chem. 2002; 277(42):39944-52. Epub 2002 Aug. 9. doi: 10.1074/jbc.M205147200. PubMed PMID: 12167620.
44. Hong S E, Shugart Y Y, Huang D T, Shahwan S A, Grant P E, Hourihane J O, Martin N D, Walsh C A. Autosomal recessive lissencephaly with cerebellar hypoplasia is associated with human RELN mutations. Nat Genet. 2000; 26(1):93-6. Epub 2000 Sep. 6. doi: 10.1038/79246. PubMed PMID: 10973257.
45. Fatemi S H, Kroll J L, Stary J M. Altered levels of Reelin and its isoforms in schizophrenia and mood disorders. Neuroreport. 2001; 12(15):3209-15. PubMed PMID: 11711858.
46. Niu S, Yabut O, D'Arcangelo G. The Reelin signaling pathway promotes dendritic spine development in hippocampal neurons. J Neurosci. 2008; 28(41):10339-48. Epub 2008 Oct. 10. doi: 10.1523/JNEUROSCI.1917-08.2008. PubMed PMID: 18842893; PMCID: PMC2572775.
47. Chen Y, Beffert U, Ertunc M, Tang T S, Kavalali E T, Bezprozvanny I, Herz J. Reelin modulates NMDA receptor activity in cortical neurons. J Neurosci. 2005; 25(36):8209-16. Epub 2005 Sep. 9. doi: 10.1523/JNEUROSCI.1951-05.2005. PubMed PMID: 16148228.
48. Qiu S, Zhao L F, Korwek K M, Weeber E J. Differential reelin-induced enhancement of NMDA and AMPA receptor activity in the adult hippocampus. J Neurosci. 2006; 26(50):12943-55. doi: 10.1523/JNEUROSCI.2561-06.2006. PubMed PMID: 17167084.
49. Qiu S, Korwek K M, Pratt-Davis A R, Peters M, Bergman M Y, Weeber E J. Cognitive disruption and altered hippocampus synaptic function in Reelin haploinsufficient mice. Neurobiol Learn Mem. 2006; 85(3):228-42. doi: 10.1016/j.nlm.2005.11.001. PubMed PMID: 16376115.
50. Seo T B, Cho H S, Shin M S, Kim C J, Ji E S, Baek S S. Treadmill exercise improves behavioral outcomes and spatial learning memory through up-regulation of reelin signaling pathway in autistic rats. J Exerc Rehabil. 2013; 9(2):220-9. Epub 2013 Nov. 28. doi: 10.12965/jer.130003. PubMed PMID: 24278864; PMCID: PMC3836510.
51. Teixeira C M, Martin E D, Sahun I, Masachs N, Pujadas L, Corvelo A, Bosch C, Rossi D, Martinez A, Maldonado R, Dierssen M, Soriano E. Overexpression of Reelin prevents the manifestation of behavioral phenotypes related to schizophrenia and bipolar disorder. Neuropsychopharmacology. 2011; 36(12):2395-405. Epub 2011 Aug. 5. doi: 10.1038/npp.2011.153. PubMed PMID: 21814183; PMCID: PMC3194085.
52. Strasser V, Fasching D, Hauser C, Mayer H, Bock H H, Hiesberger T, Herz J, Weeber E J, Sweatt J D, Pramatarova A, Howell B, Schneider W J, Nimpf J. Receptor clustering is involved in Reelin signaling. Mol Cell Biol. 2004; 24(3):1378-86. Epub 2004 Jan. 20. doi: 10.1128/mcb.24.3.1378-1386.2004. PubMed PMID: 14729980; PMCID: PMC321426.
53. Hiesberger T, Trommsdorff M, Howell B W, Goffinet A, Mumby M C, Cooper J A, Herz J. Direct binding of Reelin to VLDL receptor and ApoE receptor 2 induces tyrosine phosphorylation of disabled-1 and modulates tau phosphorylation. Neuron. 1999; 24(2):481-9. PubMed PMID: 10571241.
54. D'Arcangelo G, Homayouni R, Keshvara L, Rice D S, Sheldon M, Curran T. Reelin is a ligand for lipoprotein receptors. Neuron. 1999; 24(2):471-9. PubMed PMID: 10571240.
55. Howell B W, Herrick T M, Cooper J A. Reelin-induced tyrosine [corrected] phosphorylation of disabled 1 during neuronal positioning. Genes Dev. 1999; 13(6):643-8. Epub 1999 Mar. 25. doi: 10.1101/gad.13.6.643. PubMed PMID: 10090720; PMCID: PMC316552.
56. Howell B W, Herrick T M, Hildebrand J D, Zhang Y, Cooper J A. Dab1 tyrosine phosphorylation sites relay positional signals during mouse brain development. Curr Biol. 2000; 10(15):877-85. Epub 2000 Aug. 26. doi: 10.1016/s0960-9822(00)00608-4. PubMed PMID: 10959835.
57. Spencer C M, Alekseyenko O, Hamilton S M, Thomas A M, Serysheva E, Yuva-Paylor L A, Paylor R. Modifying behavioral phenotypes in Fmr1KO mice: genetic background differences reveal autistic-like responses. Autism Res. 2011; 4(1):40-56. Epub 2011 Jan. 27. doi: 10.1002/aur.168. PubMed PMID: 21268289; PMCID: PMC3059810.
58. Finneran D J, Morgan D, Gordon M N, Nash K R. CNS-Wide over Expression of Fractalkine Improves Cognitive Functioning in a Tauopathy Model. J Neuroimmune Pharmacol. 2018. Epub 2018 Dec. 1. doi: 10.1007/s11481-018-9822-5. PubMed PMID: 30499006.
59. Burger C, Nash K R. Small-Scale Recombinant Adeno-Associated Virus Purification. Methods Mol Biol. 2016; 1382:95-106. Epub 2015 Nov. 28. doi: 10.1007/978-1-4939-3271-9_7. PubMed PMID: 26611581.
60. Carty N, Lee D, Dickey C, Ceballos-Diaz C, Jansen-West K, Golde T E, Gordon M N, Morgan D, Nash K. Convection-enhanced delivery and systemic mannitol increase gene product distribution of AAV vectors 5, 8, and 9 and increase gene product in the adult mouse brain. J Neurosci Methods. 2010; 194(1):144-53. doi: 10.1016/j.jneumeth.2010.10.010. PubMed PMID: 20951738.
61. Castle M J, Cheng Y, Asokan A, Tuszynski M H. Physical positioning markedly enhances brain transduction after intrathecal AAV9 infusion. Sci Adv. 2018; 4(11):eaau9859. Epub 2018 Nov. 18. doi: 10.1126/sciadv.aau9859. PubMed PMID: 30443600; PMCID: PMC6235539.
62. Hudry E, Vandenberghe L H. Therapeutic AAV Gene Transfer to the Nervous System: A Clinical Reality. Neuron. 2019; 101(5):839-62. Epub 2019 Mar. 8. doi: 10.1016/j.neuron.2019.02.017. PubMed PMID: 30844402.
63. Meyer K, Ferraiuolo L, Schmelzer L, Braun L, McGovern V, Likhite S, Michels O, Govoni A, Fitzgerald J, Morales P, Foust K D, Mendell J R, Burghes A H, Kaspar B K. Improving single injection CSF delivery of AAV9-mediated gene therapy for SMA: a dose-response study in mice and nonhuman primates. Mol Ther. 2015; 23(3):477-87. Epub 2014 Nov. 2. doi: 10.1038/mt.2014.210. PubMed PMID: 25358252; PMCID: PMC4351452.
64. Hinderer C, Bell P, Katz N, Vite C H, Louboutin J P, Bote E, Yu H, Zhu Y, Casal M L, Bagel J, O'Donnell P, Wang P, Haskins M E, Goode T, Wilson J M. Evaluation of Intrathecal Routes of Administration for Adeno-Associated Viral Vectors in Large Animals. Hum Gene Ther. 2018; 29(1):15-24. Epub 2017 Aug. 16. doi: 10.1089/hum.2017.026. PubMed PMID: 28806897; PMCID: PMC5770082.
65. Nash K R, Lee D C, Hunt J B, Jr., Morganti J M, Selenica M L, Moran P, Reid P, Brownlow M, Guang-Yu Yang C, Savalia M, Gemma C, Bickford P C, Gordon M N, Morgan D. Fractalkine overexpression suppresses tau pathology in a mouse model of tauopathy. Neurobiol Aging. 2013; 34(6):1540-8. doi: 10.1016/j.neurobiolaging.2012.12.011. PubMed PMID: 23332170.
66. Nash K R, Moran P, Finneran D J, Hudson C, Robinson J, Morgan D, Bickford P C. Fractalkine over expression suppresses alpha-synuclein-mediated neurodegeneration. Mol Ther. 2015; 23(1):17-23. doi: 10.1038/mt.2014.175. PubMed PMID: 25195598; PMCID: 4426798.
67. Grossman A W, Aldridge G M, Lee K J, Zeman M K, Jun C S, Azam H S, Arii T, Imoto K, Greenough W T, Rhyu I J. Developmental characteristics of dendritic spines in the dentate gyrus of Fmr1 knockout mice. Brain Res. 2010; 1355:221-7. Epub 2010 Aug. 5. doi: 10.1016/j.brainres.2010.07.090. PubMed PMID: 20682298; PMCID: PMC3433497.
68. Grossman A W, Elisseou N M, McKinney B C, Greenough W T. Hippocampal pyramidal cells in adult Fmr1 knockout mice exhibit an immature-appearing profile of dendritic spines. Brain Res. 2006; 1084(1):158-64. Epub 2006 Apr. 1. doi: 10.1016/j.brainres.2006.02.044. PubMed PMID: 16574084.
69. Hutsler J J, Zhang H. Increased dendritic spine densities on cortical projection neurons in autism spectrum disorders. Brain Res. 2010; 1309:83-94. Epub 2009 Nov. 10. doi: 10.1016/j.brainres.2009.09.120. PubMed PMID: 19896929.
70. Irwin S A, Patel B, Idupulapati M, Harris J B, Crisostomo R A, Larsen B P, Kooy F, Willems P J, Cras P, Kozlowski P B, Swain R A, Weiler I J, Greenough W T. Abnormal dendritic spine characteristics in the temporal and visual cortices of patients with fragile-X syndrome: a quantitative examination. Am J Med Genet. 2001; 98(2):161-7. Epub 2001 Feb. 27. PubMed PMID: 11223852.
71. Li J, Pelletier M R, Perez Velazquez J L, Carlen P L. Reduced cortical synaptic plasticity and GluR1 expression associated with fragile X mental retardation protein deficiency. Mol Cell Neurosci. 2002; 19(2):138-51. Epub 2002 Feb. 28. doi: 10.1006/mcne.2001.1085. PubMed PMID: 11860268.
72. Groc L, Choquet D, Stephenson F A, Verrier D, Manzoni O J, Chavis P. NMDA receptor surface trafficking and synaptic subunit composition are developmentally regulated by the extracellular matrix protein Reelin. J Neurosci. 2007; 27(38):10165-75. Epub 2007 Sep. 21. doi: 10.1523/JNEUROSCI.1772-07.2007. PubMed PMID: 17881522; PMCID: PMC6672660.
73. Braithwaite S P, Paul S, Nairn A C, Lombroso P J. Synaptic plasticity: one STEP at a time. Trends Neurosci. 2006; 29(8):452-8. Epub 2006 Jun. 30. doi: 10.1016/j.tins.2006.06.007. PubMed PMID: 16806510; PMCID: PMC1630769.
74. Ampuero E, Jury N, Hartel S, Marzolo M P, van Zundert B. Interfering of the Reelin/ApoER2/PSD95 Signaling Axis Reactivates Dendritogenesis of Mature Hippocampal Neurons. J Cell Physiol. 2017; 232(5):1187-99. Epub 2016 Oct. 26. doi: 10.1002/jcp.25605. PubMed PMID: 27653801.
75. Selenica M-L, Benner L, Housley S B, Manchec B, Lee D C, Nash K R, Kalin J, Bergman J A, Kozikowski A, Gordon M N, Morgan D. Histone deacetylase 6 inhibition improves memory and reduces total tau levels in a mouse model of tau deposition. Alzheimers Research & Therapy. 2014; 6(1). doi: 10.1186/alzrt241. PubMed PMID: WOS:000339801600012.
76. Pujadas L, Gruart A, Bosch C, Delgado L, Teixeira C M, Rossi D, de Lecea L, Martinez A, Delgado-Garcia J M, Soriano E. Reelin regulates postnatal neurogenesis and enhances spine hypertrophy and long-term potentiation. J Neurosci. 2010; 30(13):4636-49. Epub 2010 Apr. 2. doi: 10.1523/JNEUROSCI.5284-09.2010. PubMed PMID: 20357114; PMCID: PMC6632327.
77. Eadie B D, Cushman J, Kannangara T S, Fanselow M S, Christie B R. NMDA receptor hypofunction in the dentate gyrus and impaired context discrimination in adult Fmr1 knockout mice. Hippocampus. 2012; 22(2):241-54. Epub 2010 Nov. 5. doi: 10.1002/hipo.20890. PubMed PMID: 21049485.
78. Eadie B D, Zhang W N, Boehme F, Gil-Mohapel J, Kainer L, Simpson J M, Christie B R. Fmr1 knockout mice show reduced anxiety and alterations in neurogenesis that are specific to the ventral dentate gyrus. Neurobiol Dis. 2009; 36(2):361-73. Epub 2009 Aug. 12. doi: 10.1016/j.nbd.2009.08.001. PubMed PMID: 19666116.
79. Koga K, Liu M G, Qiu S, Song Q, O'Den G, Chen T, Zhuo M. Impaired presynaptic long-term potentiation in the anterior cingulate cortex of Fmr1 knock-out mice. J Neurosci. 2015; 35(5):2033-43. Epub 2015 Feb. 6. doi: 10.1523/JNEUROSCI.2644-14.2015. PubMed PMID: 25653361; PMCID: PMC6705363.
80. Yau S Y, Bostrom C A, Chiu J, Fontaine C J, Sawchuk S, Meconi A, Wortman R C, Truesdell E, Truesdell A, Chiu C, Hryciw B N, Eadie B D, Ghilan M, Christie B R. Impaired bidirectional NMDA receptor dependent synaptic plasticity in the dentate gyrus of adult female Fmr1 heterozygous knockout mice. Neurobiol Dis. 2016; 96:261-70. Epub 2016 Oct. 19. doi: 10.1016/j.nbd.2016.09.012. PubMed PMID: 27659109.
81. Yun S H, Trommer B L. Fragile X mice: reduced long-term potentiation and N-Methyl-D-Aspartate receptor-mediated neurotransmission in dentate gyrus. J Neurosci Res. 2011; 89(2):176-82. Epub 2010 Dec. 17. doi: 10.1002/jnr.22546. PubMed PMID: 21162125.
82. Zhao M G, Toyoda H, Ko S W, Ding H K, Wu L J, Zhuo M. Deficits in trace fear memory and long-term potentiation in a mouse model for fragile X syndrome. J Neurosci. 2005; 25(32):7385-92. Epub 2005 Aug. 12. doi: 10.1523/JNEUROSCI.1520-05.2005. PubMed PMID: 16093389; PMCID: PMC6725289.
83. Paradee W, Melikian H E, Rasmussen D L, Kenneson A, Conn P J, Warren S T. Fragile X mouse: strain effects of knockout phenotype and evidence suggesting deficient amygdala function. Neuroscience. 1999; 94(1):185-92. Epub 1999 Dec. 29. doi: 10.1016/s0306-4522(99)00285-7. PubMed PMID: 10613508.
84. Martin H G S, Lassalle O, Brown J T, Manzoni O J. Age-Dependent Long-Term Potentiation Deficits in the Prefrontal Cortex of the Fmr1 Knockout Mouse Model of Fragile X Syndrome. Cereb Cortex. 2016; 26(5):2084-92. Epub 2015 Mar. 10. doi: 10.1093/cercor/bhv031. PubMed PMID: 25750254.
85. Nosyreva E D, Huber K M. Metabotropic receptor-dependent long-term depression persists in the absence of protein synthesis in the mouse model of fragile X syndrome. J Neurophysiol. 2006; 95(5):3291-5. Epub 2006 Feb. 3. doi: 10.1152/jn.01316.2005. PubMed PMID: 16452252.

What is claimed is:

1. A method of treating at least one symptom of a patient suffering from Fragile X Syndrome, comprising administering a therapeutically effective amount of a REELIN adeno-associated viral (AAV) vector expressing a secreted recombinant REELIN fusion protein, wherein the REELIN fusion protein comprises an N-terminal REELIN R3 repeat encoded by the nucleotide sequence of SEQ ID NO: 2.

2. The method of claim 1, wherein the recombinant REELIN fusion protein does not comprise a REELIN repeat R4 and R5.

3. The method of claim 1, wherein the recombinant REELIN fusion protein further comprises a C-terminal REELIN R6 repeat encoded by the nucleotide sequence of SEQ ID NO: 5.

4. The method of claim 1, wherein the recombinant REELIN fusion protein has the amino acid sequence of SEQ ID NO: 10.

5. The method of claim 4, wherein the REELIN adeno-associated viral (AAV) vector further comprises an IgKappa signal sequence fused in frame to the N-terminal REELIN R3 repeat.

6. The method of claim 4, wherein the REELIN adeno-associated viral (AAV) vector is administered intraarterially, intravenously, intracerebrally, intraventricularly or intrathecally.

7. The method of claim 4, wherein the REELIN adeno-associated viral (AAV) vector is administered by bilateral intracerebral injection.

8. The method of claim 4, wherein REELIN adeno-associated viral (AAV) vector is administered by intracerebroventricular (ICV) injection.

9. The method of claim 8, wherein the REELIN AAV vector is effective at mitigating at least one cognitive defect caused by Fragile X Syndrome.

10. The method of claim 9, wherein the mitigation of the at least one cognitive defect caused by Fragile X Syndrome is equivalent to the mitigation of the cognitive defect obtained after intracerebroventricular (ICV) injection of a R3456 REELIN repeat protein.

11. The method of claim 9, wherein the at least one cognitive defect of Fragile X Syndrome comprises hyperactivity.

12. The method of claim 9, wherein the at least one cognitive defect of Fragile X Syndrome comprises associative learning.

13. The method of claim 9, wherein the at least one cognitive defect of Fragile X Syndrome comprises spatial learning and memory.

14. The method of claim 1, wherein the symptom of Fragile X Syndrome is selected from the group consisting of a deficiency in dendritic spine density, diminished long-term potentiation, diminished synaptic plasticity and associative learning deficits.

15. The method of claim 1, wherein the recombinant REELIN fusion protein induces dimerization of an ApoER2 receptor.

16. The method of claim 1, wherein the recombinant REELIN fusion protein activates the phosphorylation of DAB1 and ERK1/2.

17. A method of treating at least one symptom of a patient suffering from Fragile X Syndrome, comprising administering a therapeutically effective amount of a REELIN adeno-associated viral (AAV) vector expressing a secreted recombinant REELIN fusion protein, wherein the fusion protein comprises an C-terminal R6 REELIN repeat encoded by the nucleotide sequence of SEQ ID NO: 5.

18. A method of treating at least one symptom of a patient suffering from Fragile X Syndrome, comprising administering a therapeutically effective amount of a REELIN adeno-associated viral (AAV) vector expressing a secreted recombinant REELIN fusion protein, wherein the REELIN fusion protein does not comprise REELIN repeats R4 or R5.
